(19) United States
(12) Patent Application Publication
(10) Pub. No.: US 2003/0162192 A1
(43) Pub. Date: Aug. 28, 2003

(54) Polymorphisms associated with ion-channel disease

(76) Inventors: John G. Sotos, Palo Alto, CA (US); Mark Edward Curran, Newark, CA (US); Marco Guida, San Diego, CA (US); Hugh Y. Rienhoff JR., San Carlos, CA (US)

Correspondence Address: SHERIDAN ROSS PC, 1560 BROADWAY, SUITE 1200, DENVER, CO, 80202

(21) Appl. No.: 10/224,683

(22) Filed: Aug. 20, 2002

Related U.S. Application Data
(60) Provisional application No. 60/314,331, filed on Aug. 20, 2001.
(60) Provisional application No. 60/378,521, filed on May 6, 2002.

(51) Int. Cl. C12Q 1/68

(52) U.S. Cl. 435/6

(57) ABSTRACT

The present invention provides methods and materials to identify genetic abnormalities that predispose an individual to ion-channel diseases. The invention provides four polymorphic sites in the KCNQ1 gene that cause reduced conductance of the associated potassium ion channel current and a variant form of the KCNE1 gene which causes decreased conductance though the channel. The variant form of KCNE1 also acts synergistically with variants of KCNQ1 to cause further decreased conductance than either variant alone. The invention further provides polymorphisms in ion channel genes showing a higher frequency in populations afflicted with ion channel diseases or within control groups. The detection of these polymorphic sites that produce the potassium ion channel protein variants in either heterozygous or homozygous form in a subject indicates that the subject has, or is susceptible to, ion channel diseases such as congenital or acquired cardiac arrhythmia, LQT syndrome, SIDS, epilepsy, or hearing loss.

### CROSS-REFERENCE TO RELATED APPLICATIONS

This application claims benefit under 35 USC §119(e) of U.S. Provisional Application No. 60/314,331, filed Aug. 20, 2001, and U.S. Provisional Application No. 60/378,521, filed May 6, 2002, which are incorporated herein in their entirety by this reference.

### FIELD OF THE INVENTION

The invention lies in the field of genetic changes associated with ion channel diseases and methods of identifying and detecting these changes in individuals having or suspected of having an ion channel disease.

### BACKGROUND OF THE INVENTION

Electrical functions in complex living organisms depend on a specialized class of molecules called “ion channels.” Ion channels are protein molecules that regulate the flow of electrically charged atoms (ions) across membranes. Complex organisms have a plurality of ion channel proteins which allow them to precisely control the timing, direction, and magnitude of ion flux (Hille, B. (1984). Ionic Channels of Excitable Membranes, pp. 99-116, Sinauer. Variations in ion flux and/or ion channel structure have been associated with several disease states, collectively referred to as “ion channel diseases.” (Schulze-Bahr, Z Kardiol 89 Suppl 4:IV12-22 (2000); Noebels News Physiol Sci. October; 13:255-256 (1998); Bockenhauer, Curr Opin Pediatr. April 2001; 13(2):142-9.; Schofield, Clin Exp Pharmacol Physiol.28(1-2):84-8. (2001)).

Examples of ion channel diseases include certain cardiac arrhythmias, epilepsy and certain other disorders of neuronal conduction, certain types of hearing loss, and certain types of muscular dysfunction. One of the earliest-discovered examples of ion channel disease is a clinical syndrome of sudden unexpected death known as the long QT syndrome (LQTS; Ward, J. Ir. Med. Assoc. 54:103-106 (1964); Romano, Lancet, 1:658-659 (1965), Jervell, Am. Heart J. 54:59-78 (1957). The name derives from the electrocardiographic characteristic of a prolonged QT interval often seen in the syndrome. Electrocardiographic recordings in humans normally show a stereotypical pattern of electrical activity in each heartbeat. Individual features of the electrocardiographic tracings of the electrical impulses have been named with a single letter, such as P, Q, R, S, and T, as illustrated in FIG. 1.

The QT interval is the length of time between the start of the QRS complex and the end of the T-wave. Upper limits of normal have been defined for the QT interval under various conditions. When the QT interval is above the upper limit of normal, LQTS is one of several possible causes (Roden, Circulation 94: 1996-2012 (1996)) including coronary heart failure, or congestive heart failure (Tomaselli, Circulation 90: 2534-2539 (1994)).

LQTS may be present as a congenital disorder or be acquired after conception. The term “acquired long QT syndrome” is often used to distinguish the acquired from the congenital forms (Karaguezian, J Cardiovasc Electrophysiol. Nov; 1(11): 1298 (2000)). Certain medications (especially cardiac anti-arrhythmics), certain dietary practices, and certain electrolyte abnormalities can precipitate acquired long QT syndrome (Zipes, Am. J. Cardiol. 59:26E-31E (1987)), Jackman, Prog Cardiovasc Dis 31: 115-172 (1988)).

Other clinical syndromes, including (but not restricted to) Brugada syndrome (Brugada, Curr Cardiol Rep. November; 2(6):507-14 (2000), sudden infant death syndrome (SIDS; Schwartz N. Engl. J. Med. 343(4):262-7 (2000), sudden unexpected death in epilepsy (SUDEP; Noebels, News Physiol. Sci.:255-256 (1998), sudden unexpected death in sleep (SUDS), and arrhythmogenic right ventricular dysplasia (ARVD; Towbin, J. Electrocardiol. 2000) may also result from abnormal ion channel function or quantity. Unfortunately, in many of these syndromes associated with acquired or congenital forms of long QT arrhythmias go undetected until a sudden unexplained death of an individual. Thus, there exists a need for a means to detect LQTS prior to an adverse cardiac event.

Certain variations in four genes involved in potassium ion flow are known to produce the long QT syndrome: KCNQ1 (also referred to as KCNQ1, KVLQT1 or LQT1), KCNH2 (also referred to as HERG or human ether-a-go-go related gene or LQT2), KCNE1 (also referred to as MinK or LQT5) and KCNE2 (also referred to as MirP1). Variation in a fifth gene, SCN5A (also referred to as hH1 or LQT3), which regulates sodium ion flow, also produces sudden unexpected death (Splawski, Circulation. September 5; 102(10): 1178-85 (2000). Collectively, these five genes are often known as the “long QT genes” or “LQT genes.” (Vincent, Ann. Med. February; 30(1):58-65 (1998)). Therefore, one means for early detection of LQTS is a test to identify genetic abnormalities that predispose an individual to ion-channel diseases.

### SUMMARY OF THE INVENTION

One embodiment of the present invention provides a method of determining an ion channel disease genotype of an individual, comprising analyzing a nucleic acid sample from the individual for the presence of a mutation indicative of decreased ion channel conductivity. The mutation may cause an amino acid change such as a lysine residue to an asparagine residue at amino acid position 393 of the KCNQ1 protein, a proline residue to an alanine residue at amino acid position 408 of the KCNQ1 protein, a proline residue to an arginine residue at amino acid position 448 of the KCNQ1 protein or a glutamic acid residue to a serine residue at amino acid position 643 of the KCNQ1 protein. The mutations causing these changes may be the substitution of a thiamine for a guanine at position 1179 of the KCNQ1 coding sequence, the substitution of a guanine for a cytosine at position 1222 of the KCNQ1 coding sequence, the substitution of a guanine for a cytosine at position 1343 of the KCNQ1 coding sequence, or the substitution of an adenine for a guanine at position 1927 of the KCNQ1 coding sequence. In another embodiment of the invention, the method may include the additional analysis of the nucleic acid sample for the presence of a mutation that results in an amino acid change from an aspartic acid residue to an asparagine residue at amino acid position 85 of the KCNE1 protein. This amino acid change may result from the substitution of an adenine for a guanine at position 671 of the KCNE1 coding sequence.

The method may include known analytical steps such as differential primer extension, allele-specific probe hybridization, allele-specific amplification, direct sequencing, denaturing gradient gel electrophoresis, and, single strand conformational polymorphism analysis.

The testing is preferentially be performed on an individual that has, or is suspected of having, an ion channel disease such as long QT syndrome, cardiac arrhythmias, epilepsy, hearing loss, SIDS, SUDEP, SUDS post-myocardial infarction complications, and acquired sudden death syndrome.

In another embodiment of the invention, the method of analyzing the nucleic acid sample of the individual includes subjecting a nucleic acid sample from the individual to amplification conditions in the presence of a pair of primers. In this embodiment, one of the primers includes at least twelve nucleotides and may have a sequence such as the sequence immediately adjacent to position 1179 of SEQ ID NO: 2 and including either a thiamine or a guanine at position 1179 of SEQ ID NO: 2 as the terminal 3′ base of the primer, the sequence immediately adjacent to position 1179 of the complement of SEQ ID NO: 2 and including either an adenine or a cytosine at position 1179 of the complement of SEQ ID NO: 2 as the terminal 3′ base of the primer, the sequence immediately adjacent to position 1222 of SEQ ID NO: 2 and including either a guanine or a cytosine at position 1222 of SEQ ID NO: 2 as the terminal 3′ base of the primer the sequence immediately adjacent to position 1222 of the complement of SEQ ID NO: 2 and including either a cytosine or a guanine at position 1222 of the complement of SEQ ID NO: 2 as the terminal 3′ base of the primer, the sequence immediately adjacent to position 1343 of SEQ ID NO: 2 and including either a guanine or a cytosine at position 1343 of SEQ ID NO: 2 as the terminal 3′ base of the primer, the sequence immediately adjacent to position 1343 of the complement of SEQ ID NO: 2 and including either a cytosine or a guanine at position 1343 of the complement of SEQ ID NO: 2 as the terminal 3′ base of the primer, the sequence immediately adjacent to position 1927 of SEQ ID NO: 2 and including either an adenine or a guanine at position 1927 of SEQ ID NO: 2 as the terminal 3′ base of the primer, the sequence immediately adjacent to position 1927 of the complement of SEQ ID NO: 2 and including either a thiamine or a cytosine at position 1927 of the complement of SEQ ID NO: 2 as the terminal 3′ base of the primer, the sequence immediately adjacent to position 671 of SEQ ID NO: 5 and including either an adenine or a guanine at position 671 of SEQ ID NO: 5 as the terminal 3′ base of the primer, or the sequence immediately adjacent to position 671 of the complement of SEQ ID NO: 5 and including either a thiamine or a cytosine at position 671 of the complement of SEQ ID NO: 5 as the terminal 3′ base of the primer.

A further embodiment of the present invention provides an isolated KCNQ1 nucleic acid molecule having the nucleic acid sequence of SEQ ID NO: 6, SEQ ID NO: 7, SEQ ID NO: 8, SEQ ID NO: 9 and/or the nucleic acid sequence that is fully complementary to these nucleic acid sequences such that the isolated nucleic acid molecule is less than about 5 kilobases in length. In other embodiment of the present invention, the isolated KCNQ1 nucleic acid molecule may be less than about 70 nucleotides in length or may be a probe of 100 or fewer nucleotides. These probes may also be conjugated to a detectable marker. These probes may also be provided as an array of oligonucleoties.

The invention also provides an isolated nucleic acid molecule having at least one base variation from that of an ion channel associated gene sequence shown in Table 4 and at least 20 other bases of the ion channel associated gene. These isolated nucleic acid molecules are less than about 5 kilobases in length.

The invention also provides an isolated nucleic acid molecule having at least one base variation from that of an ion channel associated gene sequence shown in Table 5 and at least 20 other bases of the ion channel associated gene. These isolated nucleic acid molecules are less than about 5 kilobases in length.

### BRIEF DESCRIPTION OF THE DRAWINGS

FIG. 1 shows a QT interval in an electrocardiogram.

FIG. 2 shows a current voltage relationship for cells transfected with either wt KCNQ1 or K393N KCNQ1.

FIG. 3 shows a current voltage relationship for cells tranfected with wt KCNQ1 or P408A, P448R or G643S variant forms.

FIG. 4 shows activation rates measured by tau (time constant) for wildtype and mutant forms of KCNQ1.

FIG. 5 shows deactivation rates measured by tau for wildtype and mutant forms of KCNQ1.

FIG. 6 shows current voltage relationships with cells transfected with wildtype forms of both KCNE1 and KCNQ1 compared with cells transfected with wildtype KCNQ1 and mutant KCNE1.

FIG. 7 shows KCNE1 activation rates measured by tau for cells transfected with both KCNE1 and KCNQ1 compared with cells transfected with wildtype KCNQ1 and mutant KCNE1.

FIG. 8 shows KCNE1 deactivation rates measured by tau for measured by tau for cells transfected with both KCNE1 and KCNQ1 compared with cells transfected with wildtype KCNQ1 and mutant KCNE1.

FIG. 9 shows a current voltage relationship for cells transfected with both KCNE1 and KCNQ1 compared with cells transfected with double mutant G643S KCNQ1/D85N KCNE1.

FIG. 10 shows normalized current magnitudes at 20 mV for KCNQ1 K393N, P408A, P448R and G643S and KCNE1 D85N and T125M.

FIG. 11 shows computer systems useful for storing and manipulating genetic data of the invention.

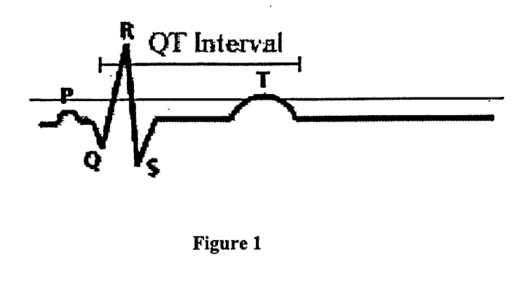

### DETAILED DESCRIPTION OF THE INVENTION

The KCNQ1 and KNCE1 genes encode protein products that associate to form a cardiac potassium ion channel although the stoichiometry has not been unequivocally defined. It has been proposed that four subunits of the KCNQ1 protein associate with four subunits of KCNE1 to form the potassium channel responsible for the cardiac current IKs (Mitchson, Cell. Phys. Biol. 9:201-216 1999). Evidence for this association comes from cotransfection experiments in which cells transfected with KCNE1 alone had no change in current flow, cells transfected with KCNQ1 alone have increased potassium current flow, and cells transfected with both KCNQ1 and KCNE1 have a markedly higher current flow than cells transfected with KCNQ1 alone, and the current mimics the native IKs as observed in cardiac myocytes. (Sanguinetti Nature 384:80-83, 1996).

The inventors have identified four polymorphic sites in the KCNQ1 gene that cause reduced conductance of the associated potassium ion channel current. The inventors have also determined that a variant form of the KCNE1 gene, which encodes a modifying subunit of the potassium ion channel, IKs and decreases conductance though the channel. The variant form also acts synergistically with variants of KCNQ1 to cause further decreased conductance than either variant alone. Detection of these polymorphic sites that produce the potassium ion channel protein variants in either heterozygous or homozygous form in a subject indicates that the subject has, or is susceptible to, ion channel diseases such as congenital or acquired cardiac arrhythmia, LQT syndrome, SIDS, epilepsy, or hearing loss. The subject can then be treated with drugs or implantable cardiac devices that ameliorate the deficiency due to the variant form of KCNQ1, counseled to avoid drugs or life situations that might exacerbate the deficiency, and/or can be regularly monitored for proper heart function. Cell lines or drugs bearing a KCNQ1 gene with one of the variant forms of the invention are useful in screening agents for pharmaceutical activity in restoring potassium ion channel conductance or for further lowering conductivity. Subjects recruited for clinical trials can also be screened for the presence or absence of the variant polymorphic forms of the invention. Certain drugs may show different efficacy/toxicity profiles depending upon whether the population does or does not have variant polymorphisms. Use of populations that are homogeneous for a given polymorphic form can facilitate detection of a statistically significant effect of a drug and allow customized selection of different drugs depending on the genetic background of a patient.

The invention provides four polymorphims in the KCNQ1 gene that occur in patients suffering from an ion channel disease, and which are shown to have variant forms correlated with decreased current through the KCNQ1/KCNE1 ion channel. All of these polymorphisms are located 3′ to the six putative transmembrane alpha helices and pore loop signature sequence of the subunit in exons 9 and 10 of the KCNQ1 gene.

SEQ ID NO: 1 is the amino acid sequence and SEQ ID NO: 2 is the coding sequence of the human KCNQ1 as described by Neyroud, Circ. Res. 84(3): 290-297 (1999) (GenBank ACCESSION AJ006345, VERSION AJ006345.1 GI:5042384). The protein has 676 amino acids. Variant proteins are described by the symbol XnY in which n is the position of an amino acid within the reference sequence, X is the amino acid occupying that position in the reference sequence and Y is the amino acid occupying that position in a variant protein. If a variant protein has a different number of amino acids than the reference protein, then the codons in the variant protein are assigned the same numbers as corresponding codons in the reference protein when the variant and reference protein are maximally aligned. Similarly, variant nucleotides within the gene are described by the symbol WnZ in which n is the position of a nucleotide within the reference sequence, W is the nucleotide occupying that position in the reference sequence and Z is the nucleotide occupying that position in a variant gene. Unless otherwise noted, the numbering used in this nomenclature within the present disclosure refers to the position of the nucleotide within the coding sequence with the adenosine nucleotide of the start ATG codon assigned nucleotide number one.

Using this nomenclature, the four polymorphisms in KCNQ1 having variant forms shown to correlate with decreased conductivity are K393N, P408A, P448R, and G643S. The identification of three of these polymorphisms is described in U.S. Provisional Patent Application No. 60/314,331. P448R is described in the same copending application and by Splawski et al., Circulation 102:1178-1185 (2000). All four of these polymorphisms have variant forms occurring in patients with an ion channel disease. The present inventors have found that the variant forms correlate with decreased current through cells expressing KCNQ1 and KCNE1 gene products indicating a causative relationship between the four identified polymorphisms and ion channel diseases.

The invention further provides a polymorphism in the KCNE1 gene encoding a subunit of the potassium ion channel. This polymorphism is referred to using analogous nomenclature to that for KCNQ1. SEQ ID NO: 3 is the amino acid sequence, SEQ ID NO: 4 is the coding sequence and SEQ ID NO: 5 is the gene sequence of the KCNE1 gene as described by Murai et al., Biochem. Biophys. Res. Commun. 161(1):176-81 (1989) (GenBank ACCESSION NM_{—}000219, VERSION NM_{—}000219.1 GI:4557686). The polymorphism is thus referred to as D85N. The polymorphism is also described by U.S. Provisional Patent Application No. 60/314,331, by George et al., WO 01/27323 and by Tesson, Mol. Cell Cardiol 28:2051-55(1996). The present inventors have found that a combination of the D85N variant form of the KCNE1 gene product with one of the four variant forms of the KCNQ1 gene product described above provides a greater reduction in current than any of the variant forms alone.

Table 1 shows the location, nucleotide change and flanking sequence of five polymorphisms of the present invention implicated in ion channel diseases. The present invention includes the sequences shown in Table 1 that comprise base changes as described herein, having appurtenant sequences of 10, 15, 20, 25, 30, 35, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, or 1000 bases, or any whole number encompassed by the range of 10-10,000.

TABLE 1

| | | | | | | |
|---|---|---|---|---|---|---|
| Poly- | Nucleotide | | SEQ ID | | Location | |
| morphism | Change | Flanking Sequence | NO | Gene | in Gene | SNP Position |

| | | | | | | |
|---|---|---|---|---|---|---|
| K 393 N | G to T | cccgactcctccacctggaa T | 6 | KCNQ1 | Exon 9 | 1179 of SEQ ID NO: 2 |
| | | atctacatccggaaggcccc | | | | |
| P 408 A | C to G | ggagccacactctgctgtca G | 7 | KCNQ1 | Exon 9 | 1222 of SEQ ID NO: 2 |
| | | ccagccccaaacccaagaag | | | | |
| P 448 R | C to G | ccatatcacgtgcgaccccc G | 8 | KCNQ1 | Exon 10 | 1343 of SEQ ID NO: 2 |
| | | agaagagcggcggctggacc | | | | |
| G 643 S | G to A | cccacatcacccagccctgc A | 9 | KCNQ1 | Exon 16 | 1927 of SEQ ID NO: 2 |
| | | gcagtggcggctccgtcgac | | | | |
| D 85 N | G to A | tcaacgtctacatcgagtcc A | 10 | KCNE1 | Exon 1 | 671 of SEQ ID NO: 2 |
| | | atgcctggcaagagaaggac | | | | |

The frequency of appearance of these polymorphisms was established within different test populations. The characteristics of the different populations sampled is shown in Table 2. All subjects were in the United States when their tissue was collected and one individual was a member of both the epilepsy group and the LQTS group. The SIDS group consisted of individuals who died from autopsy-diagnosed SIDS. Frozen thymus, brain, or liver tissue from these individuals was obtained from a tissue bank.

The epilepsy (EPIL), LQTS, and cardiac arrest (CARD) groups consisted of individuals from the Gene Trust project, managed by DNA Sciences Inc. These individuals self-reported their diagnoses and furnished blood samples to DNA Sciences Inc. The CON1 group consisted of unselected volunteers who supplied blood. Ten individuals were of Caucasian background, 10 were African-American, 6 were of Chinese background, and 6 were of Japanese background. The CON2 group consisted of unselected volunteers of several races who supplied blood for research. Several race-specific groups of otherwise unselected volunteers were also used.

TABLE 2

| Group | | Number of subjects |
|---|---|---|
| Symbol | Group Phenotype | in group |
| SIDS | Victims of sudden infant death syndrome | 122 |
| EPIL | Adults with epilepsy | 26 |
| LQTS | Adults with long QT syndrome | 4 |
| CARD | Adults survivors of cardiac arrest | 23 |
| con1 | Control population 1 - Mixed race | 32 |
| con2 | Control population 2 - Mixed race | 2270 |
| CLJW | Control population - Caucasian | 91 |
| CLJB | Control population - African-American | 95 |
| CLJH | Control population - Hispanic | 180 |
| CLJJ | Control population - Japanese-American | 77 |
| CLJC | Control population - Chinese-American | 78 |
| CLKW | Control population - Caucasian | 460 |
| CLKH | Control population - Hispanic | 89 |
| CLKB | Control population - African American | 133 |
| CLKJ | Control population - Japanese-American | 69 |
| CLKC | Control population - Chinese-American | 74 |
| CLKM | Control population - Mixed | 410 |

Table 3 shows the frequencies of wildtype and variant alleles in various populations of patients with ion channel disease or controls. As shown in Table 3, the K393N variant form was observed in SIDS individuals (0.004) and in none of the control groups. The P408A variant form was observed at a frequency of 0.004 in the SIDS group and a frequency of 0.019 in the epilepsy group but not in the control groups. The P448R variant form was found both in the SIDS group and in several of the control groups. The D85N variant form was seen with a frequency of 0.008 in the SIDS group examined, and at comparable levels in one of the control groups. The T125M variant form had a frequency of 0.12 in the SIDS group, and was absent from all other control groups studied with the exception of the control Hispanic group, in which it was found with a frequency of 0.003.

TABLE 3

| Poly- | Nucleotide | Population | Frequency of | Frequency of |
|---|---|---|---|---|
| morphism | Change | Tested | Variant Allele | Reference Allele |
| K 393 N | G1179T | SIDS | 0.004 | 0.996 |
| | | EPIL | 0.000 | 1.000 |
| | | LQTS | 0.000 | 1.000 |
| | | CARD | 0.000 | 1.000 |
| | | SIDS | 0.004 | 0.996 |
| P 408 A | C1222G | SIDS | 0.004 | 0.996 |
| | | EPIL | 0.019 | 0.981 |
| | | LQTS | 0.000 | 1.000 |
| | | CARD | 0.000 | 1.000 |
| | | SIDS | 0.008 | 0.992 |
| P 448 R | C1343G | SIDS | 0.004 | 0.996 |
| | | EPIL | 0.000 | 1.000 |
| | | LQTS | 0.000 | 1.000 |
| | | CARD | 0.000 | 1.000 |
| | | CLJW | 0.000 | 1.000 |
| | | CLJB | 0.000 | 1.000 |
| | | CLJH | 0.000 | 1.000 |
| | | CLJJ | 0.000 | 1.000 |
| | | CLJC | 0.000 | 1.000 |
| G 643 S | G1927A | SIDS | 0.004 | 0.996 |
| | | EPIL | 0.000 | 1.000 |
| | | LQTS | 0.000 | 1.000 |
| | | CARD | 0.000 | 1.000 |
| | | CLJW | 0.000 | 1.000 |
| | | CLJB | 0.043 | 1.000 |
| | | CLJH | 0.000 | 1.000 |
| | | CLJJ | 0.050 | 1.000 |
| | | CLJC | 0.055 | 1.000 |
| D 85 N | G671A | SIDS | 0.008 | 0.992 |
| | | EPIL | 0.000 | 1.000 |
| | | LQTS | 0.000 | 1.000 |
| | | CARD | 0.000 | 1.000 |
| | | CLJW | 0.000 | 1.000 |
| | | CLJB | 0.000 | 1.000 |
| | | CLJH | 0.000 | 1.000 |
| | | CLJJ | 0.007 | 0.993 |
| | | CLJC | 0.000 | 1.000 |
| T125M | C792T | SIDS | 0.012 | 0.988 |
| | | EPIL | 0.000 | 1.000 |
| | | LQTS | 0.000 | 1.000 |
| | | CARD | 0.000 | 1.000 |
| | | CLJW | 0.000 | 1.000 |
| | | CLJB | 0.000 | 1.000 |
| | | CLJH | 0.003 | 0.997 |
| | | CLJJ | 0.000 | 1.000 |

The present invention also provides novel polymorphisms found in subjects with SIDS, epilepsy, LQTS, or a history of cardiac arrest, related nucleic acid molecules (e.g. primers, probes, etc.) and nucleotides for detecting the same. Table 4 lists the polymorphism as a capitalized nucleotide, its genetic location and corresponding SEQ ID number within the ion channel genes. This includes additional polymorphisms in the KCNQ1 and KCNE1 genes as well as the HERG (GenBank ACCESSION NM_{—}000238, XM_{—}004743, AB044806), SCN5A (GenBank ACCESSION NM_{—}000335) and KCNE2 (GenBank ACCESSION NM_{—}005136, XM_{—}009744) genes.

Table 4 also shows the sequence flanking the polymorphism and the frequency with which the variant and reference alleles appear in the control or ion channel disease groups. The present invention includes the sequences shown in Table 4 that comprise base changes as described herein, having appurtenant sequences of 10, 15, 20, 25, 30, 35, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, or 1000 bases, or any whole number encompassed by the range of 10-10,000.

TABLE 4

| | Location of | Amino Acid | Nucleotide | | Polymorphism and flanking sequence | | Freq. of | Freq. of |
|---|---|---|---|---|---|---|---|---|
| Gene | Polymorphism | Change | Change | SEQID NO | (5′ to 3′) | Group tested | variant allele | reference allele |
| HERG | intron2-61 | | G to C | 11 | ggttccagggtccatcctgcCtggctttctgctctgccac | SIDS | 0.004 | 0.996 |
| | | | | | | Con1 | 0.000 | 1.000 |
| HERG | intron 2-36 | | T to A | 12 | ttctgctctgcccactgagAgggtgccaagggggctatg | SIDS | 0.004 | 0.996 |
| | | | | | | Con1 | 0.000 | 1.000 |
| HERG | exon 3 + 432 | N144N | C to T | 13 | gtggggtccccggctcatgaTaccaaccaccggggcccccc | SIDS | 0.004 | 0.996 |
| | | | | | | Con1 | 0.000 | 1.000 |
| HERG | intron3 + 78 | | C to T | 14 | tggagctgtccaaggtcaaTgctctgcaggaagggaggat | SIDS | 0.43 | 0.957 |
| | | | | | | Con1 | 0.078 | 0.922 |
| HERG | intron3 + 110 − 111 | | INST | 15 | agggaggatgtaatggttgggTtgggggtccccccttgg | SIDS | 0.017 | 0.983 |
| | | | | | | Con1 | 0.031 | 0.969 |
| HERG | intron 3 + 114 | | G to A | 16 | aggatgtaatggttgggtggAgggtcccccctttggaggtg | SIDS | 0.0004 | 0.996 |
| | | | | | | Con1 | 0.000 | 1.000 |
| HERG | intron − 70 | | C to A | 17 | cagggcctggtctccactctAgatctatgggtggcctgcct | SIDS | 0.004 | 0.996 |
| | | | | | | Con1 | 0.000 | 1.000 |
| HERG | intron 5 + 7 | | G to A | 18 | agaaggtcacccaggtaggcAcccagcggccgggctctcct | SIDS | 0.004 | 0.996 |
| | | | | | | Con1 | 0.000 | 1.000 |
| HERG | exon 6 + 1332 | Q444Q | G to A | 19 | gaaggcccgcctgctaccgaAtgtggctacgcctgccagcc | SIDS | 0.18 | 0.982 |
| | | | | | | Con1 | 0.000 | 1.000 |
| HERG | exon 6 + 1467 | I489I | C to T | 20 | gtcagccaccccggccgcatTgccgtccatacttcaaggg | SIDS | 0.192 | 0.808 |
| | | | | | | Con1 | 0.484 | 0.516 |
| HERG | exon 6 + 1539 | F513F | C to T | 21 | ccttcgacctgctcattcttTggctctggctctgaggaggt | SIDS | 0.188 | 0.183 |
| | | | | | | Con1 | 0.469 | 0.531 |
| HERG | intron 6 +111 − | | INS | 22 | cagagaaaaagagagagagaGAGAAGA | SIDS | 0.248 | 0.752 |
| | 112 | | GAGAAGA | | gagaaagagagaaagacagt | Con1 | 0.375 | 0.625 |
| HERG | intron 6 − 5 | | C to T | 23 | aatgtcccttccctgtccTccagctgatcgggctgctga | SIDS | 0.013 | 0.987 |
| | | | | | | Con1 | 0.000 | 1.000 |
| HERG | exon 7 + 1692 | L564L | G to A | 24 | gcgctcatcgcgcactggctAgcctgcatctggtacgccat | SIDS | 0.454 | 0.546 |
| | | | | | | Con1 | 0.281 | 0.719 |
| HERG | exon 7 + 1809 | G603G | C to T | 25 | tacaacagcagcggcctgggTggcccctccatcaaggacaa | SIDS | 0.029 | 0.971 |
| | | | | | | Con1 | 0.063 | 0.937 |
| HERG | exon 8 + 1956 | Y652Y | C to T | 26 | acgcccccagccctcatgtaTgctagcatcttcggcaacgt | SIDS | 0.248 | 0.752 |
| | | | | | | Con1 | 0.000 | 1.000 |
| HERG | intron 8 + 15 | | G to T | 27 | acgcggtgaggccaccagaTcgttggccagtgggtggcaggc | SIDS | 0.005 | 0.995 |
| | | | | | | Con1 | 0.000 | 1.000 |
| HERG | intron 8 + 16 | | C to T | 28 | acgcggtgaggccaccagagTgtggccagtgggtggcaggc | SIDS | 0.005 | 0.995 |
| | | | | | | Con1 | 0.000 | 1.000 |
| HERG | intron 8 + 39 | | GG to CA | 29 | gccagtgggtggcaggctgCAaagagtggggtggcagagg | SIDS | 0.343 | 0.657 |
| | | | | | | Con1 | 0.156 | 0.844 |
| HERG | intron 8 − 61 | | G to A | 30 | gaggggtgggatggtggagtAgagtgtgggttggggggtcc | SIDS | 0.291 | 0.709 |
| | | | | | | Con1 | 0.000 | 1.000 |
| HERG | exon 9 +10 2328 | L776L | C to T | 31 | gtgcatgctggggacctgctTaccgccctgtacttcatctc | SIDS | 0.004 | 0.996 |
| | | | | | | Con1 | 0.000 | 1.000 |
| HERG | exon 9 + 2371 | T791W | C to T | 32 | ggggctccatcgagatcctgTggggcgacgtcgtcgtggcc | SIDS | 0.004 | 0.996 |
| | | | | | | Con1 | 0.000 | 1.000 |
| | | | | | | Con2 | 0.000 | 1.000 |
| HERG | intron 10 − 135 | | T to C | 33 | ttgcctggggcaaaatcacaCtgggggcagggaagggtttt | SIDS | 0.004 | 0.996 |
| | | | | | | Con1 | 0.000 | 1.000 |
| HERG | intron 10 − 51 | | T to C | 34 | gggagcttggggcctgacccCggtggggcaggagagcactg | SIDS | 0.335 | 0.665 |
| | | | | | | Con1 | 0.250 | 0.750 |
| HERG | exon 11 + 2617 | G873S | G to A | 35 | acatgatccgggctcccccAgcagtacggagttagagggt | SIDS | 0.004 | 0.996 |
| | | | | | | Con1 | 0.000 | 1.000 |
| | | | | | | Con2 | 0.001 | 0.999 |
| HERG | exon 11 + 2682 | R894R | C to T | 36 | aagttgtccttccgcaggcgTacggacaagggtgaggcggg | SIDS | 0.004 | 0.996 |
| | | | | | | Con1 | 0.000 | 1.000 |
| HERG | exon 11 + 2690 | K897T | A to C | 37 | cttccgcaggcgcacggacaCgggtgaggcgggggagggg | SIDS | 0.165 | 0.835 |
| | | | | | | Con1 | 0.063 | 0.937 |
| | | | | | | CLKW | 0.248 | 0.752 |
| | | | | | | CLKH | 0.135 | 0.865 |
| | | | | | | CLKB | 0.049 | 0.951 |
| | | | | | | CLKJ | 0.072 | 0.928 |
| | | | | | | CLKC | 0.068 | 0.932 |
| | | | | | | CLKM | 0.132 | 0.868 |
| HERG | intron 11 − 57 | | C to T | 38 | cctgtcccctcctccaccctTgcccccgcctctctgttctc | SIDS | 0.252 | 0.748 |
| | | | | | | Con1 | 0.000 | 1.000 |
| HERG | intron 11 − 48 | | C to T | 39 | tcctccaccctcgccccctcTtctctgttctcctcccctct | SIDS | 0.014 | 0.986 |
| | | | | | | Con1 | 0.000 | 1.000 |
| HERG | exon 12 + 2729 | P910L | C to T | 40 | ggaggtgtcggccttggggcTgggccgggcggggcaggg | SIDS | 0.005 | 0.995 |
| | | | | | | Con1 | 0.000 | 1.000 |
| | | | | | | Con2 | 0.0003 | 1.000 |
| HERG | intron 12 + 62 | | C to T | 41 | cagcggtggtgcgtctacccTgctcacccagctctgctctc | SIDS | 0.0005 | 0.995 |
| | | | | | | Con1 | 0.000 | 1.000 |
| HERG | exon 13 + 1111 | N1037 | C to T | 42 | ggtcggcttcccggggcgaTgtggagagcaggctggatg | SIDS | 0.005 | 0.995 |
| | | | | | | Con1 | 0.000 | 1.000 |
| HERG | intron 13 + 12 | | C to A | 43 | gctaacaggtgagggagtgAaggtggggtgggggggac | SIDS | 0.009 | 0.991 |
| | | | | | | Con1 | 0.000 | 1.000 |
| HERG | intron 12 + 22 | | G to A | 44 | tgagggagtgcaggtggggtAggggggcacgccctggagtc | SIDS | 0.234 | 0.776 |
| | | | | | | Con1 | 0.359 | 0.641 |
| HERG | intron 13 + 65 | | G to A | 45 | gtcaggtcctggcggtgttAtctggtagagggagagggcc | SIDS | 0.005 | 0.995 |
| | | | | | | Con1 | 0.000 | 1.000 |
| HERG | intron 13 − 15 | | C to T | 46 | ccacttctctgagcatccccTacttcctgccccaggctgga | SIDS | 0.004 | 0.996 |
| | | | | | | Con1 | 0.000 | 1.000 |
| HERG | exon 14 + 3162 | T1054T | C to G | 47 | tcctccccaggctggagacGcggctgagtgcagacatggc | SIDS | 0.004 | 0.996 |
| | | | | | | Con1 | 0.000 | 1.000 |
| HERG | intron 14 − 8 − 9 | | Del Gt | 48 | cgcctgcccatgctctgtgt--attgcaggtttcccagttca | SIDS | 0.004 | 0.996 |
| | | | | | | Con1 | 0.031 | 0.969 |
| HERG | 3′UTR + 112 | | C to T | 49 | cgtggaaggggagaggaactTgaaagcacagctcctcccc | SIDS | 0.034 | 0.966 |
| | | | | | | Con1 | 0.047 | 0.953 |
| HERG | 3′UTR + 190 | | C to T | 50 | cccagtgagaggggcaggggTagggccggcagtaggtggg | SIDS | 0.009 | 0.991 |
| | | | | | | Con1 | 0.000 | 1.000 |
| SCN5A | exon 2 + 87 | A29A | G to A | 51 | gccatcgagaagcgcatggcAgagaagcaagcccgcggctc | SIDS | 0.149 | 0.851 |
| SNC5A | exon 2 + 100 | R34C | C to T | 52 | gcatggcggagaagcaagccTgcggctcaacaccttgcag | SIDS | 0.074 | 0.926 |
| SCN5A | exon 2 + 141 | P47P | C to T | 53 | gagagccgagaggggcgcggTgaggaggaggctcccctgg | SIDS | 0.004 | 0.996 |
| SCN5A | intron 2 + 51 | | G to A | 54 | cccttctgtgcaactccttAtcaa | SIDS | 0.058 | 0.942 |
| SCN5A | intron 2 − 25 | | C to T | 55 | caagcctttctacacaagggTctaatgctacatgtctgtcc | SIDS | 0.112 | 0.888 |
| SCN5A | intron 2 − 26 | | G to A | 56 | ccaagcctttctacacaaggAcctaatgctacatgtctgtc | SIDS | 0.069 | 0.931 |
| SCN5A | exon 3 + 354 | | C to T | 57 | tatgtcctcagtcccttccaTcccatccggagagcggctgt | SIDS | 0.009 | 0.991 |
| SCN5A | intron 4 + 16 | | G to C | 58 | gtcgagtgagtatcttcaggCcctcttctccagtggcccc | SIDS | 0.030 | 0.970 |
| SCN5A | exon 5 + 486 | | C to T | 59 | cggcctctcctgcccaggtaTcccatccggagagcggctgt | SIDS | 0.004 | 0.996 |
| SCN5A | intron 6 + 45 | | C to A | 60 | gctagagggatatgcctgggActttccagccacctgggagc | SIDS | 0.031 | 0.969 |
| SCN5A | intron 6 − 68 | | Del C | 61 | accagctaactcaggcccag-cccccaccagtggagcacag | SIDS | 0.005 | 0.995 |
| SCN5A | intron 6 − 43 | | C to T | 62 | caccagtggagcacagagctTggtgcccctgggtgaccccg | SIDS | 0.005 | 0.995 |
| SCN5A | exon 7 + 717 | | C to T | 63 | tccccagggctgaagaccatTgtgggggccctgatccagtc | SIDS | 0.005 | 0.995 |
| SCN5A | exon 7 + 856 | A286T | G to A | 64 | agtgcgtgcgcaacttcaca-Acgtcaacggcaccaacggc | SIDS | 0.004 | 0.996 |
| SCN5A | exon 9 + 1068 | | T to C | 65 | cacggctacaccagcttcgaCtcctttgcctgggcctttct | SIDS | 0.008 | 0.992 |
| SCN5A | intron 9 + 3 | | C to A | 66 | cctctgcccccttgctccccAagaccctcaggtccgcaggg | SIDS | 0.142 | 0.858 |
| SCN5A | exon 10 + 1302 | | C to T | 67 | gaggagaaggaaaagcgcttTcaggaggccatggaaatgct | SIDS | 0.018 | 0.982 |
| SCN5A | exon 11 + 1381 | L461V | T to G | 68 | ataccgtgtcccgtagctccGtggagatgtcccctttggcc | SIDS | 0.008 | 0.992 |
| SCN5A | exon 12 + 1569 | | T to A | 69 | caggacttctatgaagcccgAtccagccgcgggagcatttt | SIDS | 0.004 | 0.996 |
| SCN5A | exon 12 + 1571 | S524Y | C to A | 70 | ggacttctatgaagccgttAcagccgcgggagcattttca | SIDS | 0.009 | 0.991 |
| SCN5A | exon 12 +10 1587 | | T to C | 71 | cgttccagccgcgggagcatCttcacctttcgcaggcgaga | SIDS | 0.004 | 0.996 |
| SCN5A | exon 12 + 1673 | H558R | A to G | 72 | gggggagagcgagaccaccGcacatcacctgctggtgccc | SIDS | 0.241 | 0.759 |
| SCN5A | exon 12 + 1743 | | G to A | 73 | cagcccagtcccggaacctcAgctcctggccagcctcca | SIDS | 0.004 | 0.996 |
| SCN5A | exon 12 + 1852 | L618F | C to T | 74 | ccacatccccaggaagccacTtcctccgccctgtgatgcta | SIDS | 0.004 | 0.996 |
| SCN5A | exon 12 + 1889 | T630M | C to T | 75 | gctagagcacccgccagacaTggtgagccagccccgagatg | SIDS | 0.004 | 0.996 |
| SCN5A | intron 12 + 14 | | G to A | 76 | agacaggtgagccagccccAagatgcaggcaccacagca | SIDS | 0.009 | 0.991 |
| SCN5A | exon 13 + 1967 | P656L | C to T | 77 | tgtagatggcttcgaggagcTaggagcacggcagcgggccc | SIDS | 0.009 | 0.991 |
| SCN5A | exon 14 + 2066 | R689H | G to A | 78 | gtgtccaccatgctggaaccAtctcgcccagcgctacctga | SIDS | 0.004 | 0.996 |
| SCN5A | intron 14 + 33 | | Del G | 79 | tgcagctggctctcaaatgg-catcctggcaggaccccacc | SIDS | 0.022 | 0.978 |
| SCN5A | intron 15 + 12 | | G to A | 80 | cttccgctggtacctggctgAacccactgcatgggggatgg | SIDS | 0.004 | 0.996 |
| SCN5A | intron 16 + 6 | | C to T | 81 | ggtgagcctgaccattatctTgacaggtcctgaatctcttc | SIDS | 0.038 | 0.962 |
| SCN5A | exon 17 + 3183 | | G to A | 82 | gacacagatgaccaagaagaAgatgaggagaacagcctggg | SIDS | 0.120 | 0.880 |
| SCN5A | intron 17 + 30 | | G to A | 83 | accctctggctgggtgtgtgAactcagctataggctggg | SIDS | 0.008 | 0.992 |
| SCN5A | exon 18 + 3249 | | C to T | 84 | caggaatcccagcctgtgtcTggtggcccagaggcccctcc | SIDS | 0.004 | 0.996 |
| SCN5A | exon 18 + 3308 | S1103Y | C to A | 85 | ccaggtgtcagcgactgcctActctgaggccgaggccagtg | SIDS | 0.059 | 0.941 |
| SCN5A | exon 18 + 3319 | E1107K | G to A | 86 | cgactgcctcctctgaggccAaggccagtgcatctcaggcc | SIDS | 0.004 | 0.996 |
| SCN5A | exon 19 + 3392 | T1131I | C to T | 87 | cccacacccctgtccatagaTcccagaggacagttgctccg | SIDS | 0.004 | 0.996 |
| SCN5A | intron 19 + 24 | | C to T | 88 | atggcccgggcagccctctgTcctagcctcagttccacca | SIDS | 0.004 | 0.996 |
| SNC5A | exon 20 + 3578 | R1193Q | G to A | 89 | cccaggaaggtctggtggcAgttgcgcaagacctgctacc | SIDS | 0.009 | 0.991 |
| SCN5A | exon 20 − 3621 | | C to T | 90 | atctggagcacagctggttTgagacattcatcattcttcat | SIDS | 0.004 | 0.996 |
| SCN5A | intron 20 − 9 | | C to T | 91 | agtggagcgctggtaccctcTtggggatgcagggttgtggc | SIDS | 0.004 | 0.996 |
| SCN5A | intron 20 − 35 | | C to T | 92 | atgcagggttgtggcagggaTggctggaggaggaggggag | SIDS | 0.004 | 0.996 |
| SCN5A | intron 20 + 6 | | A to C | 93 | aggaggaggggagggcagggCaaggaggtctccagcgtgg | SIDS | 0.004 | 0.996 |
| SCN5A | intron 20 + 67 − 68 | | Ins AA | 94 | ggggagggcagggaaaggAAaggtctccagcgtggaag | SIDS | 0.004 | 0.996 |
| SCN5A | exon 23 + 4218 | | G to A | 95 | tcaactttgacaacgtgggAgccgggtacctggcccttct | SIDS | 0.014 | 0.986 |
| SCN5A | intron 24 + 28 | | C to T | 96 | gccacagtggcttcttcaacTaagtcaggcacctgaggctc | SIDS | 0.995 | |
| SCN5A | intron 24 − 38 − 45 | | Del | 97 | cttcttccaccaagtcaggc------ctcctggttgcttggccacc | SIDS | 0.029 | 0.971 |
| | | | ACCTGAGG | | | | | |
| SCN5A | intron 24 + 53 | | T to C | 98 | caggaccctgaggctcctggCtgctggccaccagggaatc | SIDS | .0.95 | 0.905 |
| SCN5A | exon 26 + 4509 | | C to T | 99 | gccatgaagaagctgggctcTaagaagccccagaagcccat | SIDS | 0.013 | 0.987 |
| SCN5A | exon 28 + 4856 | | C to T | 100 | gacatcatccagaagtacttTttctccccgacgctcttccg | SIDS | 0.093 | 0.907 |
| SCN5A | exon 28 + 4870 | V1624I | G to A | 101 | tctccccgacgctcttccgaAtcatccgcctggcccgaata | SIDS | 0.004 | 0.996 |
| SCN5A | exon 28 + 5457 | | C to T | 102 | gtcctgtctgactttgccgaTgccctgtctgagccactccg | SIDS | 0.496 | 0.504 |
| SCN5A | exon 28 + 5711 | S1904L | C to T | 103 | gcgcaagacgaagaggtgtTggccatggttatccagagag | SIDS | 0.005 | 0.995 |
| SCN5A | exon 28 + 5844 | | C to T | 104 | cctgagcgagagggcctcatTgcctacgtgatgagtgagaa | SIDS | 0.103 | 0.897 |
| SCN5A | exon 28 + 6010 | F2004L | T to C | 105 | acagtgaagatctcgccgacCtccccccttctccggacagg | SIDS | 0.005 | 0.995 |
| KCNQ1 | aternate exon 1 | P5L | C to T | 106 | tggcactggtgc Tgggcctggattt | SIDS | 0.004 | 0.996 |
| | + 96 | | | | | SIDS | 0.004 | 0.996 |
| | | | | | | EPIL | 0.000 | 1.000 |
| | | | | | | LQTS | 0.000 | 1.000 |
| | | | | | | CARD | 0.000 | 1.000 |
| | | | | | | CLJW | 0.000 | 1.000 |
| | | | | | | CLJB | 0.000 | 1.000 |
| | | | | | | CLJH | 0.000 | 1.000 |
| | | | | | | CLJJ | 0.000 | 1.000 |
| | | | | | | CLJC | 0.000 | 1.000 |
| KCNQ1 | Intron 1A + 14 | | C to T | 107 | tcgccgtgtgagtatcgccaTcggcgacggccggacgaag | SIDS | 0.004 | 0.996 |
| KCNQ1 | Intron 1B − 17 | | G to A | 108 | aggccgtgatgctgactgccAtgtccctgtcttgcagcttc | SIDS | 0.013 | 0.987 |
| KCNQ1 | exon 2 + 447 | A149A | C to T | 109 | tccaccatcgagcagtatgcTgccctggccacggggatct | SIDS | 0.004 | 0.996 |
| KCNQ1 | Intron 2 + 9 | | C to T | 110 | ctcttctggatggtacgtagTatctgagggcatggctggat | SIDS | 0.025 | 0.975 |
| KCNQ1 | intron 2 − 10 | | G to A | 111 | gcgtcccactctAtccctgcaggag | SIDS | 0.052 | 0.948 |
| | | | | | | EPIL | 0.000 | 1.000 |
| | | | | | | LQTS | 0.000 | 1.000 |
| | | | | | | CARD | 0.000 | 1.000 |
| KCNQ1 | intron 3 − 24 | | C to T | 112 | gatcacgaaaagTtccccctctcct | SIDS | 0.058 | 0.942 |
| | | | | | | EPIL | 0.000 | 1.000 |
| | | | | | | LQTS | 0.000 | 1.000 |
| | | | | | | CARD | 0.000 | 1.000 |
| KCNQ1 | exon 5 + 720 | H240H | C to T | 113 | cagatcctgaggatgctacaTgtcgaccgccagggaggac | SIDS | 0.005 | 0.995 |
| KCNQ1 | intron 5 + 39 | | G to C | 114 | gggtgcggggcccaggttggCgacaggacggagggagcag | SIDS | 0.005 | 0.995 |
| KCNQ1 | intron 8 − 97 | | C to T | 115 | tgccacccagaggggaggggTcaggcctggggaacaggga | SIDS | 0.004 | 0.996 |
| KCNQ1 | intron 9 + 87 | | A to G | 116 | cagcacgaggctgggatctcGccatgcatttggcttggtac | SIDS | 0.008 | 0.992 |
| KCNQ1 | intron 10 + 29 | | G to A | 117 | cagttgggggccAcggggccgggaa | SIDS | 0.000 | 1.000 |
| | | | | | | EPIL | 0.000 | 1.000 |
| | | | | | | LQTS | 0.00 | 1.000 |
| | | | | | | CARD | 0.025 | 0.975 |
| KCNQ1 | intron 10 − 41 | | G to T | 118 | actggcaggttgTgtgggaggccta | SIDS | 0.012 | 0.988 |
| | | | | | | EPIL | 0.000 | 1.000 |
| | | | | | | LQTS | 0.000 | 1.000 |
| | | | | | | CARD | 0.000 | 1.000 |
| KCNQ1 | intron 10 − 41 | | G to C | 119 | actggcaggttgCgtgggaggccta | SIDS | 0.004 | 0.996 |
| | | | | | | EPIL | 0.000 | 1.000 |
| | | | | | | LQTS | 0.000 | 1.000 |
| | | | | | | CARD | 0.000 | 1.000 |
| KCN1 | intron 10 − 39 | | T to G | 120 | tggcaggttgggGgggaggcctaac | SIDS | 0.174 | 0.826 |
| | | | | | | EPIL | 0.111 | 0.889 |
| | | | | | | LQTS | 0.000 | 1.000 |
| | | | | | | CARD | 0.119 | 0.881 |
| KCNQ1 | intron 10 31 27 | | C to T | 121 | tgggaggcctaaTgtgctgtcccca | SIDS | 0.008 | 0.992 |
| | | | | | | EPIL | 0.000 | 1.000 |
| | | | | | | LQTS | 0.000 | 1.000 |
| CARD | 0.000 | 1.000 | | | | | | |
| KCNQ1 | intron 10 − 14 | | C to T | 122 | gtgctgtccccaTactttctcctca | SIDS | 0.008 | 0.992 |
| | | | | | | EPIL | 0.019 | 0.981 |
| | | | | | | LQTS | 0.000 | 1.000 |
| | | | | | | CARD | 0.071 | 0.929 |
| KCNQ1 | exon 11 + 1455 | F485F | C to T | 123 | aaccaacagcttTgccgaggacctg | SIDS | 0.029 | 0.971 |
| | | | | | | EPIL | 0.000 | 1.000 |
| | | | | | | LQTS | 0.000 | 1.000 |
| | | | | | | CARD | 0.000 | 1.000 |
| KCNQ1 | intron 11 − 46 | | A to G | 124 | ggaggggactggGgctcaaggagtc | SIDS | 0.660 | 0.340 |
| | | | | | | EPIL | 0.352 | 0.648 |
| | | | | | | LQTS | 0.333 | 0.667 |
| | | | | | | CARD | 0.500 | 0.500 |
| KCNQ1 | intron 11 − 31 | | C to G | 125 | acagggtggccaGtcacaatctcct | SIDS | 0.064 | 0.936 |
| | | | | | | EPIL | 0.000 | 1.000 |
| | | | | | | LQTS | 0.000 | 1.000 |
| | | | | | | CARD | 0.000 | 1.000 |
| KCNQ1 | intron 12 + 14 | | T to C | 126 | taagccctgtgcCgagccttcctgc | SIDS | 0.076 | 0.924 |
| | | | | | | EPIL | 0.100 | 0.900 |
| | | | | | | LQTS | 0.000 | 1.000 |
| | | | | | | CARD | 0.045 | 0.955 |
| KCNQ1 | exon 13 + 1638 | S546S | G to A | 127 | tgagcagtactcAcagggccacctc | SIDS | 0.142 | 0.858 |
| | | | | | | EPIL | 0.212 | 0.788 |
| | | | | | | LQTS | 0.000 | 1.000 |
| | | | | | | CARD | 0.238 | 0.762 |
| KCNQ1 | intron 113 + 21 | | G to A | 128 | ggccaaacggcaAcggggagggtgc | SIDS | 0.004 | 0.996 |
| | | | | | | EPIL | 0.000 | 1.000 |
| | | | | | | LQTS | 0.000 | 1.000 |
| | | | | | | CARD | 0.000 | 1.000 |
| KCNQ1 | intron 13 + 36 | | G to A | 129 | gggagggtgcccAggtcctgcccag | SIDS | 03.52 | 0.648 |
| | | | | | | EPIL | 0.308 | 0.692 |
| | | | | | | LQTS | 0.125 | 0.875 |
| | | | | | | CARD | 0.429 | 0.571 |
| KCNQ1 | intron 13 − 49 | | C to T | 130 | acagtgcatctgTgcagtgccaggg | SIDS | 0.054 | 0.946 |
| | | | | | | EPIL | 0.000 | 1.000 |
| | | | | | | LQTS | 0.000 | 1.000 |
| | | | | | | CARD | 0.000 | 1.000 |
| KCNQ1 | intron 14 − 53 | | T to C | 131 | gcccagctggggCccccggcccacc | SIDS | 0.050 | 0.950 |
| | | | | | | EPIL | 0.000 | 1.000 |
| | | | | | | LQTS | 0.000 | 1.000 |
| | | | | | | CARD | 0.000 | 1.000 |
| KCNQ1 | intron 15 + 32 | | G to T | 132 | tgcggtggttctTgttagcgtcctg | SIDS | 0.033 | 0.967 |
| | | | | | | EPIL | 0.074 | 0.926 |
| | | | | | | LQTS | 0.000 | 1.000 |
| | | | | | | CARD | 0.068 | 0.932 |
| KCNQ1 | exon 16 + 1986 | Y662Y | C to T | 133 | cctgcccacctaTgagcagctgacc | SIDS | 0.185 | 0.815 |
| | | | | | | EPIL | 0.100 | 0.900 |
| | | | | | | LQTS | 0.000 | 1.000 |
| | | | | | | CARD | 0.238 | 0.762 |
| KCNQ1 | exon 16: | | G to T | 134 | caataccccatgTaccatgctgtct | SIDS | 0.008 | 0.992 |
| | 3′UTR + 139 | | | | | EPIL | 0.000 | 1.000 |
| | | | | | | LQTS | 0.000 | 1.000 |
| | | | | | | CARD | 0.000 | 1.000 |
| KCNQ1 | exon 16: | | T to C | 135 | cacatggtgatgCtgacatcactgg | SIDS | 0.004 | 0.996 |
| | 3′UTR + 264 | | | | | EPIL | 0.019 | 0.981 |
| | | | | | | LQTS | 0.000 | 1.000 |
| | | | | | | CARD | 0.045 | 0.955 |
| KCNQ1 | exon 16: | | G to A | 136 | cacaggctgagtAcaggcccaccct | SIDS | 0.004 | 0.996 |
| | | | | | | EPIL | 0.000 | 1.000 |
| | | | | | | LQTS | 0.000 | 1.000 |
| | | | | | | CARD | 0.000 | 1.000 |
| KCNQ1 | exon 16: | | Del G | 137 | caccctgcttggccccaggggagacagag | SIDS | 0.022 | 0.978 |
| | 3′UTR + 377 | | | | | | | |
| KCNQ1 | exon 16: | | C to T | 138 | aacccctggaccTcagcctcaaatc | SIDS | 0.025 | 0.975 |
| | 3′UTR + 411 | | | | | EPIL | 0.023 | 0.977 |
| | | | | | | LQTS | 0.000 | 1.000 |
| | | | | | | CARD | 0.029 | 0.971 |
| KCNQ1 | exon 16: | | C to T | 139 | gacagagcaacccctggaccTcagcctcaaatccaggacc | SIDS | 0.052 | 0.948 |
| | 3′UTR + 411 | | | | | | | |
| KCNQ1 | exon 16: | | G to A | 140 | gcagggcaggaccagcccacActgactacagggccgccgg | SIDS | 0.004 | 0.996 |
| | 3′UTR + 464 | | | | | | | |
| KCNQ1 | exon 16: | | G to A | 141 | gactacagggccAccggcaataaaa | SIDS | 0.092 | 0.908 |
| | 3′UTR + 479 | | | | | EPIL | 0.135 | 0.865 |
| | | | | | | LQTS | 0.000 | 1.000 |
| | | | | | | CARD | 0.059 | 0.941 |
| KCNQ1 | exon 16: | | G to A | 142 | cccacgctgactacagggccAccggcaataaaagcccagga | SIDS | 0.104 | 0.896 |
| | 3′UTR + 479 | | | | | | | |
| KCNQ1 | exon 16: | | G to A | 143 | tacagggccgccAgcaataaaagcc | SIDS | 0.048 | 0.952 |
| | 3′UTR + 482 | | | | | EPIL | 0.000 | 1.000 |
| | | | | | | LQTS | 0.000 | 1.000 |
| | | | | | | CARD | 0.026 | 0.974 |
| KCNQ1 | exon 16: | | G to A | 144 | acgctgactacagggccgccAgcaataaagcccaggagcc | SIDS | 0.061 | 0.939 |
| | 3′UTR + 482 | | | | | | | |
| KCNQ1 | exon 16: | | G to A | 145 | agcccactgtgcAtggggctccgc | SIDS | 0.000 | 1.000 |
| | 3′UTR + 731 | | | | | EPIL | 0.019 | 0.981 |
| | | | | | | LQTS | 0.000 | 1.000 |
| | | | | | | CARD | 0.000 | 1.000 |
| KCNQ1 | exon 16: | | G to A | 146 | cgtggggctccAcctccaaccct | SIDS | 0.050 | 0.950 |
| | 3′UTR + 742 | | | | | EPIL | 0.000 | 1.000 |
| | | | | | | LQTS | 0.000 | 1.000 |
| | | | | | | CARD | 0.023 | 0.977 |
| KCNQ1 | exon 16: | | G to A | 147 | cagagaagtgacAgttcctacacag | SIDS | 0.004 | 0.996 |
| | 3′UTR + 837 | | | | | EPIL | 0.000 | 1.000 |
| | | | | | | LQTS | 0.000 | 1.000 |
| | | | | | | CARD | 0.000 | 1.000 |
| KCNQ1 | exon 16: | | A to G | 148 | tctgggcattacGtgccatagaaat | SIDS | 0.368 | 0.632 |
| | 3′UTR + 875 | | | | | EPIL | 0.346 | 0.654 |
| | | | | | | LQTS | 0.000 | 1.000 |
| | | | | | | CARD | 0.318 | 0.682 |
| KCNQ1 | exon 16:) | | T to C | 149 | aatttgtggtgaCttggatctgtgt | SIDS | 0.000 | 1.000 |
| | | | | | | EPIL | 0.109 | 0.981 |
| | | | | | | LQTS | 0.000 | 1.000 |
| | | | | | | CARD | 0.000 | 1.000 |
| KCNQ1 | exon 16: | | A to G | 150 | aatgagtttcacGgtgtgattttga | SIDS | 0.364 | 0.636 |
| | 3′UTR + 932 | | | | | EPIL | 0.340 | 0.660 |
| | | | | | | LQTS | 0.000 | 1.000 |
| | | | | | | CARD | 0.318 | 0.682 |
| KCNQ1 | exon 16: | | C to T | 151 | tttcctaataaaTgtggagaatcac | SIDS | 0.008 | 0.992 |
| | 3′UTR + 975 | | | | | EPIL | 0.000 | 1.000 |
| | | | | | | LQTS | 0.000 | 1.000 |
| | | | | | | CARD | 0.000 | 1.000 |
| KCNEI | Promoter-89 | | C to T | 152 | tgtatacgtgtgtgtgcgaTgtgtgggttttggccaatac | SIDS | 0.009 | 0.991 |
| KCNEI | Promter-91 | | T to C | 153 | attgtatacgtgtgtgtgcgCagtgtgggttttggccaat | SIDS | 0.018 | 0.982 |
| KCNEI | Promoter-160 | | G to A | 154 | acagacccaagatggcacacAccatggcctgtggagtgtca | SIDS | 0.036 | 0.964 |
| KCNEI | Promoter-161 | | C to T | 155 | cacagacccaagatggcacaTgccatggcctgtggagtgtc | SIDS | 0.308 | 0.692 |
| KCNEI | Promoter-188 | | G to A | 156 | ccattcatctatcaagagaaAgcattgcacagacccaagat | SIDS | 0.013 | 0.986 |
| KCNEI | Promoter-225 | | G to T | 157 | tggagtggtggatggaaataTaagggaatcaatgtatccat | SIDS | 0.004 | 0.996 |
| KCNEI | Promoter-320 | | A to G | 158 | aaaccaaaatgcacacatgcGacccatacgccacaatatg | SIDS | 0.540 | 0.460 |
| KCNEI | Promoter-374 | | C to T | 159 | catgaggaaaaagaaggagaTtgaatgagttggatgacctg | SIDS | 0.018 | 0.982 |
| KCNEI | Promoter-481 | | G to A | 160 | gaatgcacacaccagcaccAcaagtgagccacaaaagcac | SIDS | 0.348 | 0.652 |
| KCNEI | Promoter-478 | | G to A | 161 | gaaggcagagaaaagaagtgAgtgttcaccatgggttgtat | SIDS | 0.027 | 0.973 |
| KCNEI | Promoter-622 | | C to T | 163 | ctgactagtcttgcataagcTgccaggaactagttgtatga | SIDS | 0.161 | 0.839 |
| KCNEI | Promoter-747 | | C to T | 164 | agacattctgaagtccaccaTgtgacagcatagatttttca | SIDS | 0.004 | 0.996 |
| KCNEI | exon 1 + 112 | S38G | A to G | 165 | tccccccgcagcGgtgacggcaagc | SIDS | 0.300 | 0.700 |
| KCNEI | exon 1 + 374 | T125M | C to T | 166 | accttcctgagaTgaagccttcccc | SIDS | 0.012 | 0.988 |
| | | | | | | EPIL | 0.000 | 1.000 |
| | | | | | | LQTS | 0.000 | 1.000 |
| | | | | | | CARD | 0.000 | 1.000 |
| | | | | | | CLJW | 0.000 | 1.000 |
| | | | | | | CLJB | 0.000 | 1.000 |
| | | | | | | CLJH | 0.003 | 0.997 |
| | | | | | | CLJJ | 0.000 | 1.000 |
| | | | | | | CLJC | 0.000 | 1.000 |
| KCNE2 | Promoter-614 | | T to C | 167 | taaaatgcttctttcaaatcAcatagtacacacccctccct | SIDS | 0.019 | 0.981 |
| KCNE2 | Promoter-552 | | G to T | 168 | gccacttagacataggggaTgaacaaattggagagtctgt | SIDS | 0.010 | 0.990 |
| KCNE2 | Promoter-180 | | A to G | 169 | atataaatatcaaagtgtatGtataattttctctgccat | SIDS | 0.005 | 0.995 |
| KCNE2 | Promoter-89 | | A to G | 170 | tgtacgcagtcagtttaaagGctaacaaaatatgcattaaa | SIDS | 0.005 | 0.995 |
| KCNE2 | exon 1:5′UTR | | C to T | 171 | agccaaatccagaaaagatcTgttttcctaaccttgttcgc | SIDS | 0.319 | 0.681 |
| KCNE2 | exon 1:5′UTR | | A to G | 172 | ttaaccttgttcgcctattttGttatttaaattgcagcagga | SIDS | 0.128 | 0.872 |
| KCNE2 | exon 1 + 22 | T8A | A to G | 173 | tgtctactttatccatttcGcacagacgctggaagacgtc | SIDS | 0.004 | 0.996 |
| KCNE2 | exon 1 + 228 | | A to G | 175 | agcactgtgaaatccaagagGcgggaacactccaatgaccc | SIDS | 0.004 | 0.996 |
| KCNE2 | exon1: | | G to A | 176 | ctaacatctgacAtccagacatgaa | SIDS | 0.004 | 0.996 |
| | 3′UTR + 33 | | | | | EPIL | 0.000 | 1.000 |
| | | | | | | LQTS | 0.000 | 1.000 |
| | | | | | | CARD | 0.000 | 1.000 |

The present invention also provides novel polymorphisms found within the genes associated with long QT syndrome in subjects with no known disease related nucleic acid molecules (e.g. primers, probes, etc.) and nucleotides for detecting the same. As discussed below, such polymorphisms are useful in a variety of applications. Table 5 lists the polymorphism as a capitalized nucleotide, its genetic location, the corresponding SEQ ID number and the sequence flanking the polymorphism. Table 5 also shows the frequency with which the variant and reference alleles appear within the control group. The present invention includes the sequences shown in Table 5 that comprise base changes as described herein, having appurtenant sequences of 10, 15, 20, 25, 30, 35, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, or 1000 bases, or any whole number encompassed by the range of 10-10,000.

TABLE 5

| | Location | Amino | Nucleo- | | | | Freq. of | Freq. of |
|---|---|---|---|---|---|---|---|---|
| | of Poly- | Acid | tide | | Polymorphism and flanking sequence | Group | variant | reference |
| Gene | morphism | Change | Change | SEQID NO | (5′ to 3′) | tested | allele | allele |
| HERG | exon 6 + | P486P | C to T | | aggaggtggtcagccacccTggccgcatcgccgtccactac | Con1 | 0.016 | 0.984 |
| | 1458 | | | | | | | |
| HERG | intron 7 + | | C to T | | gtgtgcccaggggcgggcggTggggagagcccacggtggag | Con1 | 0.016 | 0.984 |
| | 25 | | | | | | | |
| HERG | intron 11 + | | G to A | | gggaggaagggggagggcggAgacaaggtgaggctgggagc | Con1 | 0.234 | 0.766 |
| | 35 | | | | | | | |
| HERG | intron 12 + | | C to T | | ctgtcaggtatcccgggcgaTgggcgggcgagggaggaccg | Con1 | 0.016 | 0.984 |
| | 14 | | | | | | | |
| HERG | exon 14 + | P1076P | C to T | | agatgacgctggtcccgccTgcctacagtgctgtgaccacc | Con1 | 0.016 | 0.984 |
| | 3228 | | | | | | | |
| HERG | exon 15 + | | G to A | | agttagtggggctgcccagtAtggacacgtggctcacccag | Con1 | 0.016 | 0.984 |
| | 15 | | | | | | | |
| HERG | exon 15 + | | T to A | | tcccccagcccttgggaccaActtctcctgcagtcccctgg | Con1 | 0.016 | 0.984 |
| | 147 | | | | | | | |
| HERG | exon 15 + | | C to T | | aaggacttttctgctatttaTtgctcttattgttaaggata | Con1 | 0.016 | 0.984 |
| | 311 | | | | | | | |
| HERG | exon 15 + | | T to C | | tgaataataaataattatccCgaggagactccagtggtgct | Con1 | 0.016 | 0.984 |
| | 397 | | | | | | | |

### Analysis of Polymorphisms

Polymorphisms are detected in a target nucleic acid from an individual being analyzed. For assay of genomic DNA, virtually any biological sample (other than pure red blood cells) is suitable. “Tissue” means any sample taken from any subject, preferably a human. For example, convenient tissue samples include whole blood, semen, saliva, tears, urine, fecal material, sweat, buccal epithelium, skin and hair. For assay of cDNA or mRNA, the tissue sample must be obtained from an organ in which the target nucleic acid is expressed.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated herein in its entirety by this reference for all purposes).

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The term “patient” refers to both human and veterinary subjects. The term “subject” or “individual” typically refers to humans, but also to mammals and other animals, multicellular organisms such as plants, and single-celled organisms or viruses. The identity of bases occupying the polymorphic sites shown in Table 4 can be determined in an individual (e.g., a patient being analyzed) by several methods, which are described as follows:

1. Single Base Extension Methods

Single base extension methods are described by e.g., U.S. Pat. No. 5,846,710, U.S. Pat. No. 6,004,744, U.S. Pat. No. 5,888,819 and U.S. Pat. No. 5,856,092. In brief, the methods work by hybridizing a primer that is complementary to a target sequence such that the 3′ end of the primer is immediately adjacent to, but does not span a site of, potential variation in the target sequence. That is, the primer comprises a subsequence from the complement of a target polynucleotide terminating at the base that is immediately adjacent and 5′ to the polymorphic site. The term primer refers to a single-stranded oligonucleotide capable of acting as a point of initiation of template-directed DNA synthesis under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer but typically ranges from 15 to 40 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified. Hybridization probes are capable of binding in a base-specific manner to a complementary strand of nucleic acid. Such probes include nucleic acids and peptide nucleic acids as described in Nielsen et al., Science 254, 1497-1500 (1991). A probe primer can be labeled, if desired, by incorporating a label detectable by spectroscopic, photochemical, biochemical, immunochemical, or chemical means. For example, useful labels include 32P, fluorescent dyes, electron dense reagents, enzymes (as commonly used in an ELISA), biotin, or haptens and proteins for which antisera or monoclonal antibodies are available. A label can also be used to “capture” the primer, so as to facilitate the immobilization of either the primer or a primer extension product, such as amplified DNA, on a solid support. The hybridization is performed in the presence of one or more labeled nucleotides complementary to base(s) that may occupy the site of potential variation. For example, for biallelic polymorphisms, two differentially labeled nucleotides can be used. For tetraallelic polymorphisms, four differentially-labeled nucleotides can be used. In some methods, particularly methods employing multiple differentially labeled nucleotides, the nucleotides are dideoxynucleotides. Hybridization is performed under conditions permitting primer extension if a nucleotide complementary to a base occupying the site of variation if the target sequence is present. Extension incorporates a labeled nucleotide thereby generating a labeled extended primer. If multiple differentially-labeled nucleotides are used and the target is heterozygous then multiple differentially-labeled extended primers can be obtained. Extended primers are detected providing an indication of which base(s) occupy the site of variation in the target polynucleotide.

2. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent such that there is a significant difference in hybridization intensity between alleles, and preferably an essentially binary response, whereby a probe hybridizes to only one of the alleles. Hybridizations are usually performed under stringent conditions that allow for specific binding between an oligonucleotide and a target DNA containing one of the polymorphic sites shown in Table 4. Stringent conditions are defined as any suitable buffer concentrations and temperatures that allow specific hybridization of the oligonucleotide to highly homologous sequences spanning at least one of the polymorphic sites shown in Table 4 and any washing conditions that remove non-specific binding of the oligonucleotide. For example, conditions of 5×SSPE (750 mM NaCl, 50 mM Na Phosphate, 5 mM EDTA, pH 7.4) and a temperature of 25-30° C. are suitable for allele-specific probe hybridizations. The washing conditions usually range from room temperature to 60° C. Some probes are designed to hybridize to a segment of target DNA such that the polymorphic site aligns with a central position (e.g., in a 15 mer at the 7 position; in a 16 mer, at either the 8 or 9 position) of the probe. This probe design achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. The polymorphisms can also be identified by hybridization to nucleic acid arrays, some examples of which are described by WO 95/11995 (incorporated by this reference in its entirety for all purposes).

3. Allele-Specific Amplification Methods

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer that hybridizes at a distal site. Amplification proceeds from the two primers leading to a detectable product signifying that the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, e.g., WO 93/22456. In other methods, a double-base mismatch is used in which the first mismatch is included in the 3′-most position of the oligonucleotide aligned with the polymorphism and a second mismatch is positioned at the immediately adjacent base (the pen-ultimate 3′ position). This double mismatch further prevents amplification in instances in which there is no match between the 3′ position of the primer and the polymorphism.

4. Direct-Sequencing

The direct analysis of the sequence of polymorphisms of the present invention can be accomplished using either the dideoxy-chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures that are partially dependent upon the base sequence. The different electrophoretic mobilities of single-stranded amplification products can be related to base-sequence differences between alleles of target sequences.

### Methods of Use

After determining polymorphic form(s) present in an individual at one or more polymorphic sites, this information can be used in a number of methods.

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. By analogy, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

The polymorphisms shown in Table 4 can be analyzed for a correlation with an ion channel disease as well as with response to drugs used to treat these diseases.

Correlation is performed for a population of individuals who have been tested for the presence or absence of an ion channel disease or an intermediate phenotype and for one or more polymorphic markers. To perform such analysis, the presence or absence of a set of polymorphic forms (i.e. a polymorphic set) is determined for a set of the individuals, some of whom exhibit a particular trait, and some of which exhibit lack of the trait. The alleles of each polymorphism of the set are then reviewed to determine whether the presence or absence of a particular allele is associated with the trait of interest. Correlation can be performed by standard statistical methods including, but not limited to, chi-squared test, Analysis of Variance, parametric linkage analysis, non-parametric linkage analysis, etc. and statistically significant correlations between polymorphic form(s) and phenotypic characteristics are noted. For example, it might be found that the presence of allele A1 at polymorphism A correlates with an ion channel disease. As a further example, it might be found that the combined presence of allele A1 at polymorphism A and allele B 1 at polymorphism B correlates with an ion channel disease.

Polymorphic forms that correlate with an ion channel disease are also useful in diagnosing ion channel diseases or susceptibility thereto. Combined detection of several such polymorphic forms typically increases the probability of an accurate diagnosis. For example, the presence of a single polymorphic form known to correlate with an ion channel disease might indicate a probability of 20% that an individual has or is susceptible to an ion channel disease, whereas detection of five polymorphic forms, each of which correlates with less than 20% probability, might indicate a probability up to 80% that an individual has or is susceptible to an ion channel disease. Analysis of the polymorphisms of the invention can be combined with that of other polymorphisms or other risk factors of an ion channel disease, such as family history. Polymorphisms can be used to diagnose an ion channel disease at the pre-symptomatic stage, as a method of post-symptomatic diagnosis, as a method of confirmation of diagnosis or as a post-mortem diagnosis.

Patients diagnosed with an ion channel disease can be treated with conventional therapies and/or can be counseled to avoid environmental factors and drugs that exacerbate the condition or trigger episodes. Conventional therapies for ion channel diseases include, but are not limited to, implantable devices, beta-adrenergic antagonists, avoidance of electrolyte abnormalities and certain medications, and the avoidance of certain physical activities such as swimming. Patients diagnosed with ion channel disease may also be counseled about the risk of genetically transmitting the disease to offspring, or counseled about the risk of family members sharing genetic variation(s) relevant to ion channel disease.

The polymorphic forms of the invention are useful for screening agents for either beneficial or harmful activity to patients with ion channel disease. In general, a beneficial activity is one that increases the conductance of the KCNQ1 potassium channel thus counteracting the effect of the variant forms in decreasing conductance. Agents with such an activity are useful for prophylactic or therapeutic treatment of patients that have or are susceptible to ion channel disease. In general, a harmful activity is one that decreases the conductance of the KCNQ1 potassium channel thus agonizing the effect of the variant forms in decreasing conductance. Although some such agents may have a useful therapeutic effect in addition to decreasing conductance, their use should in general be avoided in patients having one or more of the polymorphic forms of the invention.

Drug screening assays can be performed on cells that have been transfected with a nucleic acid encoding a KCNQ1 and/or KCNE1 subunits. Preferably, no endogenous equivalents of transfected nucleic acids are present in the cells. The cells can be transfected with RNA in which case expression of KCNQ1 and/or KCNE1 is transient. Alternatively, KCNQ1 and/or KCNE1 can be stably introduced into the cell line. Cells expressing KCNQ1 and/or KCNE1 are monitored for conductance and/or ion flux between the inside and outside of the cell in the presence of a test agent relative to a control. The control can be vehicle without an agent or can be an agent known not to have any effect on the KCNQ1/KCNE1 ion channel. Additionally, the control could be a known agonist and/or antagonist of Iks thereby assuring that the correct current is being monitored. An increase in conductance or ion flux responsive to administration of agent is indicative of an antagonizing effect, and a decrease in conductance is indicative of an agonizing effect. Transfected cells are also useful for identifying genes whose expression pattern is altered in the presence of variant forms of KCNQ1/KCNE1 relative to wildtype form. Such genes themselves are potential therapeutic or diagnostic targets for heart conditions.

Drug screening assays can also be performed on transgenic animals. Some transgenic animals have an exogenous human transgene bearing a variant form of KCNQ1 and/or KCNE1 of the invention. In some such animals, the endogenous equivalent(s) of transfected gene(s) transgene is/are knocked out. In other transgenic animals, the endogenous KCNQ1 or KCNE1 gene is mutated to contain one of the variant forms of the present invention. Potential agents are administered to transgenic animal, and performance of the heart is monitored (e.g., rate, EGK, QT interval). Optionally, the performance can be compared with that of a transgenic animal administered a control substance or with a nontransgenic animal administered the agent or a control substance. Agents that affect the performance of the heart (in either direction) relative to a control have a potentially useful pharmacological activity. Also agents that affect the performance of the heart (in either direction) relative to a control, which are intended for therapeutic use for some unrelated indication, are indicated as having potential side effects on the heart, signaling that such an agent should be avoided or monitored in certain patients (e.g., those with heart conditions).

Agents for screening can be obtained by producing and screening large combinatorial libraries. Combinatorial libraries can be produced for many types of compound that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes). Peptide libraries can also be generated by phage display methods. See, e.g., Devlin, WO 91/18980. The libraries of compounds can be initially screened for specific binding to the KCNQ1 or KCNE1 proteins. Preferred agents bind with a Kd<μM. For example, for receptor ligand combinations, the assay can be performed using cloned receptor immobilized to a support such as a microtiter well and binding of compounds can be measured in competition with ligand to the receptor. Agonist or antagonist activity can then be assayed using a cellular reporter system or a transgenic animal model.

The polymorphisms of the invention are also useful for conducting clinical trials of drug candidates for ion channel disease. Such trials are performed on treated or control populations selected to have or lack one or more of the variant polymorphic forms of the invention. For example, populations can be selected in which each member is hetero- or homozygous for at least one of the variant polymorphic forms K393N, P408A, P448R, and G643S and D85N. Alternatively, populations can be selected that are homozygous for the wildtype form at all five of the above polymorphisms. Use of genetically matched populations eliminates or reduces variation in treatment outcome due to genetic factors, leading to a more accurate assessment of the efficacy of a potential drug and of the genetic population on which it is effective.

Furthermore, the polymorphic forms of the invention may be used after the completion of a clinical trial to elucidate differences in response to a given treatment. For example, one or more of the variant polymorphic forms can be used to stratify the enrolled patients into disease sub-types or classes. The variant polymorphic forms of the invention can also be used to identify subsets of patients with similar polymorphic profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the polymorphic forms can be used to identify the genetic factors involved in adverse response to treatment (adverse events). For example, patients who show adverse response may have more similar polymorphic profiles than would be expected by chance. This allows the early identification and exclusion of such individuals from treatment. It also provides information that can be used to understand the biological causes of adverse events and to modify the treatment to avoid such outcomes.

The polymorphism(s) showing the strongest correlation with ion channel diseases within a given gene are likely either to have a causative role in the manifestation of the phenotype or to be in linkage disequilibrium with the causative variants. Such a role can be confirmed by in vitro gene expression of the variant gene or by producing a transgenic animal expressing a human gene bearing such a polymorphism and determining whether the animal develops an ion channel disease. Polymorphisms in coding regions that result in amino acid changes usually cause an ion channel disease by decreasing, increasing or otherwise altering the activity of the protein encoded by the gene in which the polymorphism occurs. Polymorphisms in coding regions that introduce stop codons usually cause an ion channel disease by reducing (heterozygote) or eliminating (homozygote) functional protein produced by the gene. Occasionally, stop codons result in production of a truncated peptide with aberrant activities relative to the full-length protein. Polymorphisms in regulatory regions typically cause an ion channel disease by causing increased or decreased expression of the protein encoded by the gene in which the polymorphism occurs. Polymorphisms in intronic or untranslated sequences can cause an ion channel disease either through the same mechanism as polymorphisms in regulatory sequences or by causing altered splicing patterns resulting in an altered protein.

The precise role of polymorphisms in the genes shown in Table 4 can be elucidated by several means. Alterations in expression levels of a protein can be determined by measuring protein levels in sample groups of persons characterized as having or not having an ion channel disease (or intermediate phenotypes). Alterations in ion channel activity can similarly be detected by assaying for ion channel activity in samples from the above groups of persons.

Having identified certain polymorphisms as having causative roles in an ion channel disease, and having elucidated, at least in general terms, whether such polymorphisms increase or decrease the activity or expression level of associated proteins, customized therapies can be devised for classes of patients with different genetic subtypes of metabolic diseases. For example, if a polymorphism in a given protein causes an ion channel disease by increasing the expression level or activity of the protein, the diseases associated with the polymorphism can be treated by administering an antagonist of the protein. If a polymorphism in a given protein causes ion channel disease by decreasing the expression level or activity of a protein, the form of an ion channel disease associated with the polymorphism can be treated by administering the protein itself, a nucleic acid encoding the protein that can be expressed in a patient, or an analog or agonist of the protein. This is most likely accomplished via the administration of an agent that forces the ion channel into an open conformation (i.e. for potassium channels having decreased function) or the administration of an ion channel blocking agent (i.e. for some SCN5A mutations).

Agonists and antagonists can be obtained by producing and screening large combinatorial libraries. Combinatorial libraries can be produced for many types of compounds that can be synthesized in a step by step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated herein by this reference for all purposes). Peptide libraries can also be generated by phage display methods. See, e.g., Devlin, WO 91/18980. The libraries of compounds can be initially screened for specific binding to the protein for which agonists or antagonists are to be identified, or to its natural binding ligand. Preferred agents bind with a Kd<1 μM. For example, for receptor ligand combinations, the assay can be performed using a cloned receptor immobilized to a support such as a microtiter well and binding of compounds can be measured in competition with ligand to the receptor. Agonist or antagonist activity can then be assayed using a cellular reporter system or a transgenic animal model.

The polymorphisms of the invention are also useful for conducting clinical trials of drug candidates for ion channel diseases. Such trials are performed on treated or control populations having similar or identical polymorphic profiles at a defined collection of polymorphic sites. Use of genetically matched populations eliminates or reduces variation in treatment outcome due to genetic factors, leading to a more accurate assessment of the efficacy of a potential drug.

Furthermore, the polymorphisms of the invention may be used after the completion of a clinical trial to elucidate differences in response to a given treatment. For example, the set of polymorphisms may be used to stratify the enrolled patients into disease sub-types or classes. It may further be possible to use the polymorphisms to identify subsets of patients with similar polymorphic profiles who have unusual (high or low) response to treatment or who do not respond at all (non-responders). In this way, information about the underlying genetic factors influencing response to treatment can be used in many aspects of the development of treatments (these range from the identification of new targets, through the design of new trials to product labeling and patient targeting). Additionally, the polymorphisms may be used to identify the genetic factors involved in adverse response to treatment (adverse events). For example, patients who show adverse response may have more similar polymorphic profiles than would be expected by chance. This would allow the early identification and exclusion of such individuals from treatment. It would also provide information that might be used to understand the biological causes of adverse events and to modify the treatment to avoid such outcomes.

The polymorphic DNA sequences of the present invention listed in Table 4 can also be used to prepare probes or as primers for detection of the presence of the long QT genes. In this manner, the presence of these genes can be detected from biological samples isolated from an individual of interest. This allows the presence of these genes to be assayed in selected patients. Additionally, the sequences listed in Tables 1 and 4 that have been found to reside within the coding regions of these genes can be used to assay a biological sample from an individual for the presence of gene expression by detection of the corresponding mRNA transcript. Using detection means known to those of skill in the art, these sequences of the present invention can also be used to evaluate quantitative expression of these genes as it may differ between individuals or within different tissues in the same individual.

The reported polymorphisms may also be in linkage disequilibrium with nearby genes (within 30 kb or greater) that are not related to ion channel diseases, but contribute to phenotypes such as autoimmune diseases, inflammation, cancer, diseases of the nervous system, and infection by pathogenic microorganisms. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). The more sites that are analyzed the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are diallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. The term genotype as used herein broadly refers to the genetic composition of an organism, including, for example, whether a diploid organism is heterozygous or homozygous for one or more alleles of interest. In diallelic loci, four genotypes are possible: AA, AB, BA, and BB. If alleles A and B occur in a haploid genome of the organism with frequencies x and y, the probability of each genotype in a diploid organism can be calculated as described in International Publication WO 95/12607 which is incorporated herein by this reference in its entirety. These calculations can be extended for any number of polymorphic forms at a given locus. For example, in a locus of n alleles, the appropriate binomial expansion is used to calculate p(ID) and p(exc).

If several polymorphic loci are tested, the cumulative probability of non-identity for random individuals becomes very high (e.g., one billion to one). Such probabilities can be taken into account together with other evidence in determining the guilt or innocence of the suspect.

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the putative father, it can be concluded, barring experimental error, that the putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father) can be calculated as described in International Publication WO 95/12607 which is incorporated herein by this reference in its entirety.

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

Linkage describes the tendency of genes, alleles, loci or genetic markers to be inherited together as a result of their location on the same chromosome, and can be measured by percent recombination between the two genes, alleles, loci or genetic markers that are physically-linked on the same chromosome. Loci occurring within 50 centimorgan of each other are linked. Some linked markers occur within the same gene or gene cluster.

Linkage disequilibrium (LD) or allelic association means the preferential association of a particular allele or genetic marker with a specific allele, or genetic marker at a nearby chromosomal location more frequently than expected by chance for any particular allele frequency in the population. For example, if locus X has alleles a and b, which occur with equal frequency, and linked locus Y has alleles c and d, which occur with equal frequency, one would expect the haplotype ac to occur with a frequency of 0.25 in a population of individuals. If ac occurs more frequently, then alleles a and c are considered in linkage disequilibrium. Linkage disequilibrium may result from natural selection of a certain combination of alleles or because an allele has been introduced into a population too recently to have reached equilibrium (random association) between linked alleles.

A marker in linkage disequilibrium with disease predisposing variants can be particularly useful in detecting susceptibility to disease (or association with sub-clinical phenotypes) notwithstanding that the marker does not cause the disease. For example, a marker (X) that is not itself a causative element of a disease, but which is in linkage disequilibrium with a gene (including regulatory sequences) (Y) that is a causative element of a phenotype, can be used to indicate susceptibility to the disease in circumstances in which the gene Y may not have been identified or may not be readily detectable. Younger alleles (i.e., those arising from mutation relatively late in evolution) are expected to have a larger genomic segment in linkage disequilibrium. The age of an allele can be determined from whether the allele is shared among different human ethnic groups and/or between humans and related species.

The polymorphisms shown in Table 4 can also be used to establish physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Landau et al., Proc. Natl. Acad. Sci. (USA) 83, 7353—7357 (1986); Landau et al., Proc. Natl. Acad. Sci. (USA) 84, 2363-2367 (1987); Donis-Keller et al., Cell 51, 319-337 (1987); Landau et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992) (each of which is incorporated herein by this reference in its entirety for all purposes).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of lod (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is; probability of data if loci linked at θ to probability of data if loci unlinked. The computed likelihoods are usually expressed as the log10 of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction. Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

### Modified Polypeptides and Gene Sequences

The invention further provides variant forms of nucleic acids and corresponding proteins. The nucleic acids comprise one of the sequences described in Table 4 in which the polymorphic position is occupied by an alternative base for that position. Some nucleic acids encode full-length variant forms of proteins. Similarly, variant proteins have the prototypical amino acid sequences encoded by a nucleic acid sequence shown in Table 4 (read so as to be in-frame with the full-length coding sequence of which it is a component) except at an amino acid encoded by a codon including one of the polymorphic positions shown in the Table. That position is occupied by the amino acid coded by the corresponding codon in the alternative forms shown in Table 4.

Variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer that is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like.

The protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

The invention further provides transgenic nonhuman animals capable of expressing an exogenous variant gene and/or having one or both alleles of an endogenous variant gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. Inactivation of endogenous variant genes can be achieved by forming a transgene in which a cloned variant gene is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide useful drug screening systems.

In addition to substantially full-length polypeptides expressed by variant genes, the present invention includes biologically active fragments of the polypeptides, or analogs thereof, including organic molecules that simulate the interactions of the peptides. Biologically active fragments include any portion of the full-length polypeptide that confers a biological function on the variant gene product, including ligand binding and antibody binding. Ligand binding includes binding by nucleic acids, proteins or polypeptides, small biologically active molecules or large cellular structures.

Polyclonal and/or monoclonal antibodies that specifically bind to variant gene products but not to corresponding prototypical gene products are also provided. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

### Kits

The invention further provides kits comprising at least one allele-specific oligonucleotide as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting any or all of the polymorphisms shown in Table 4. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

### Computer Systems for Storing Polymorphism Data

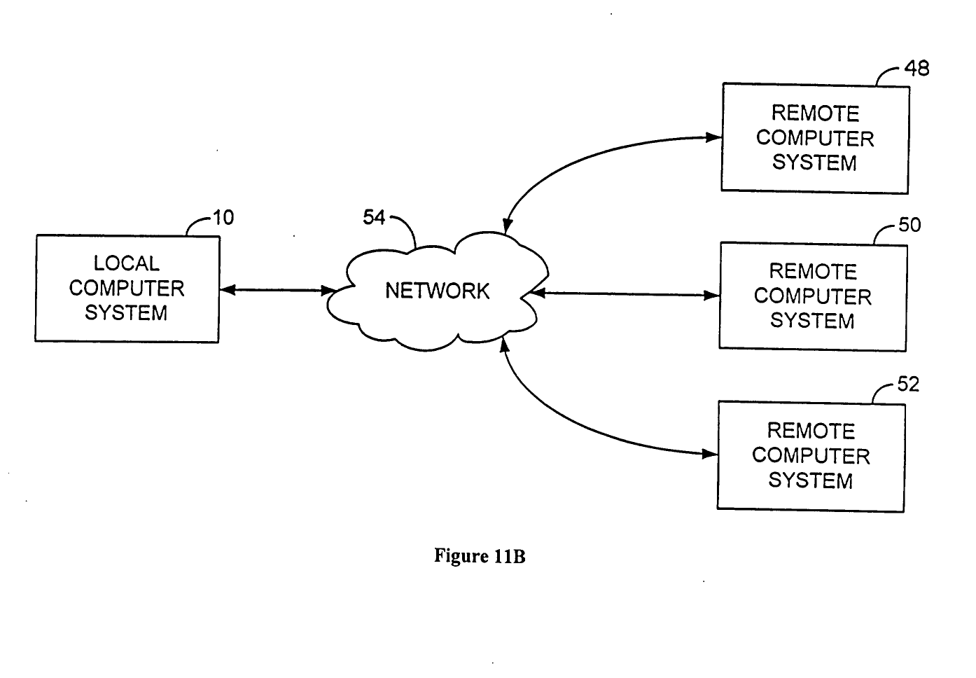

FIG. 11A depicts a block diagram of a computer system 10 suitable for implementing the present invention. Computer system 10 includes a bus 12 which interconnects major subsystems such as a central processor 14, a system memory 16 (typically RAM), an input/output (I/O) controller 18, an external device such as a display screen 24 via a display adapter 26, serial ports 28 and 30, a keyboard 32, a fixed disk drive 34 via a storage interface 35 and a floppy disk drive 36 operative to receive a floppy disk 38, and a CD-ROM (or DVD-ROM) device 40 operative to receive a CD-ROM 42. Many other devices can be connected such as a user pointing device, e.g., a mouse 44 connected via serial port 28 and a network interface 46 connected via serial port 30.

Many other devices or subsystems (not shown) may be connected in a similar manner. Also, it is not necessary for all of the devices shown in FIG. 11 to be present to practice the present invention, as discussed below. The devices and subsystems may be interconnected in different ways from that shown in FIG. 11. The operation of a computer system such as that shown in FIG. 11 is well known. Databases storing polymorphism information according to the present invention can be stored, e.g., in system memory 16 or on storage media such as fixed disk 34, floppy disk 38, or CD-ROM 42. An application program to access such databases can be operably disposed in system memory 16 or sorted on storage media such as fixed disk 34, floppy disk 38, or CD-ROM 42.

FIG. 11 depicts the interconnection of computer system 10 to remote computers 48, 50, and 52. FIG. 11 depicts a network 54 interconnecting remote servers 48, 50, and 52. Network interface 46 provides the connection from client computer system 10 to network 54. Network 54 can be, e.g., the Internet. Protocols for exchanging data via the Internet and other networks are well known. Information identifying the polymorphisms described herein can be transmitted across network 54 embedded in signals capable of traversing the physical media employed by network 54.

Information identifying polymorphisms shown in the tables above is represented in records, which optionally, are subdivided into fields. Each record stores information relating to a different polymorphism. Collectively, the records can store information relating to all of the polymorphisms in the tables above, or any subset thereof, such as 5, 10, 50, or 100 polymorphisms from Table 2. In some databases, the information identifies a base occupying a polymorphic position and the location of the polymorphic position. The base can be represented as a single letter code (i.e., A, C, G or T/U) present in a polymorphic form other than that in the reference allele. Alternatively, the base occupying a polymorphic site can be represented in IUPAC ambiguity code. The location of a polymorphic site can be identified as its position within one of the sequences shown in the tables. For example, in the first sequence shown in Table 4, the polymorphic site occupies the G or C base. The position can also be identified by reference to, for example, a chromosome, and distance from known markers within the chromosome. In other databases, information identifying a polymorphism contains sequences of 10-100 bases or the complements thereof, including a polymorphic site of the present invention. Preferably, such information records at least 10, 15, 20, or 30 contiguous bases of sequences including a polymorphic site.

All publications and patent applications cited above are incorporated by reference in their entirety for all purposes to the same extent as if each individual publication or patent application were specifically and individually indicated to be so incorporated by reference. Although the present invention has been described in some detail by way of illustration for purposes of clarity and understanding, it will be apparent that certain changes and modifications may be practiced within the scope of the appended claims.

The following Examples are provided to illustrate embodiments of the present invention and are not intended to limit the scope of the invention as set forth in the claims.

### EXAMPLES

Wild-type expression constructs for KCNQ1 and KCNE1 were obtained from Dr. Michael Sanguinetti of the University of Utah. Using standard site directed mutagenesis techniques, expression constructs were generated for the following variants: KCNQ1-K393N, KCNQ1-P408A, KCNQ1-P448A, KCNQ1-G643S, KCNE1-D85N, and KCNE1-T125M. RNA was synthesized for each construct and Xenopus oocytes injected with 6 ng of KCNQ1 cRNA and 0.6 ng KCNE1 cRNA for analysis by whole cell voltage clamp techniques.

### Example 1

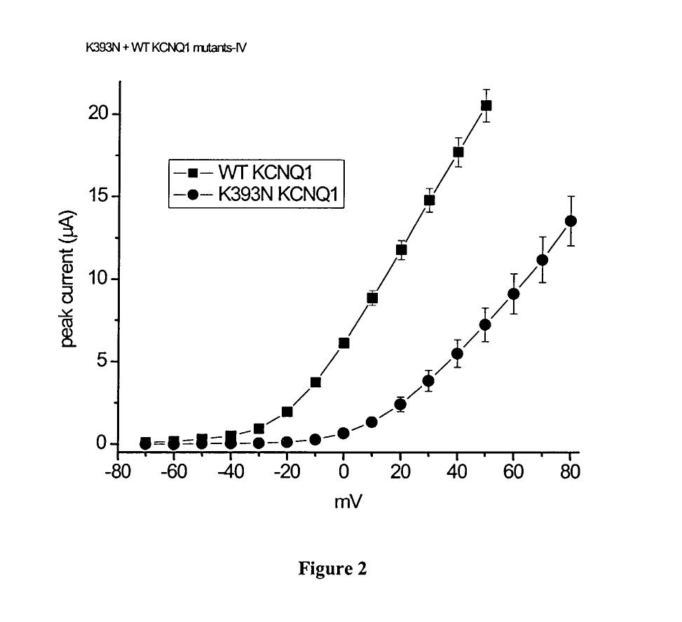

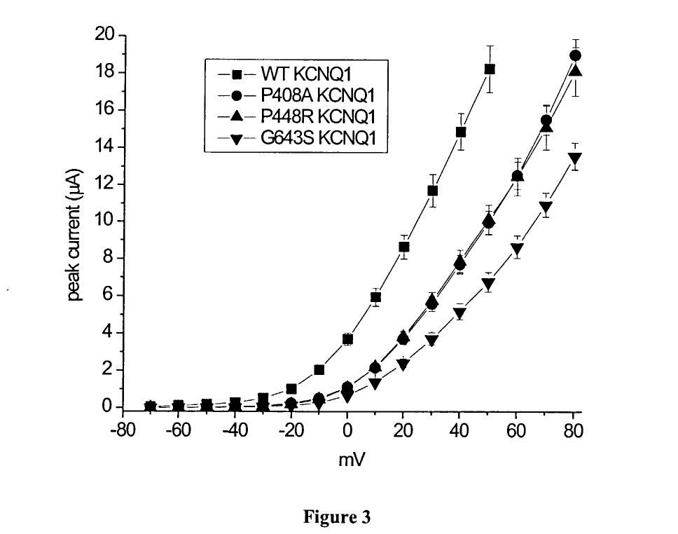

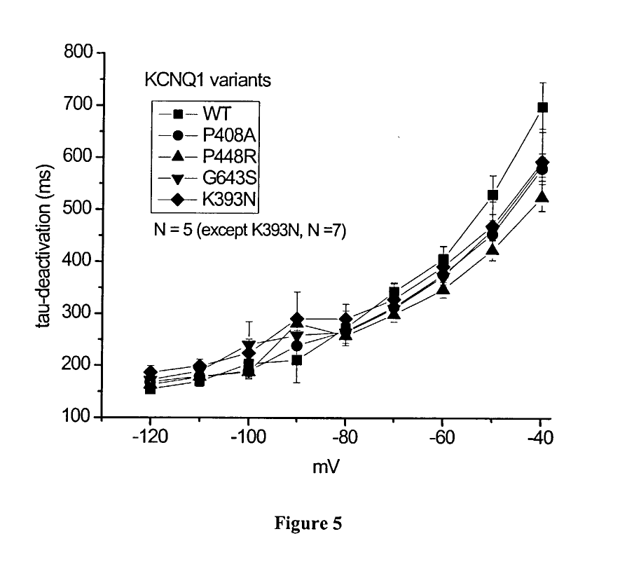

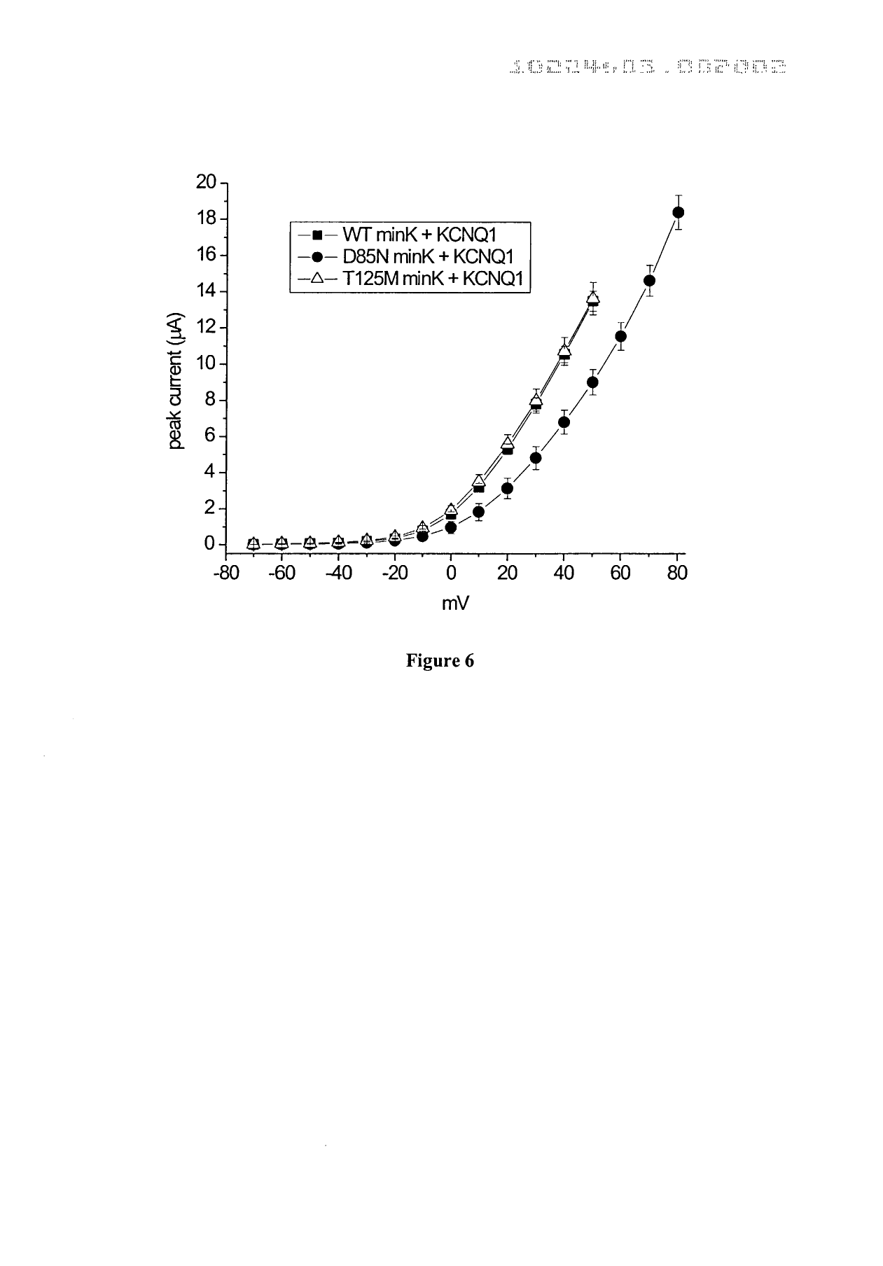

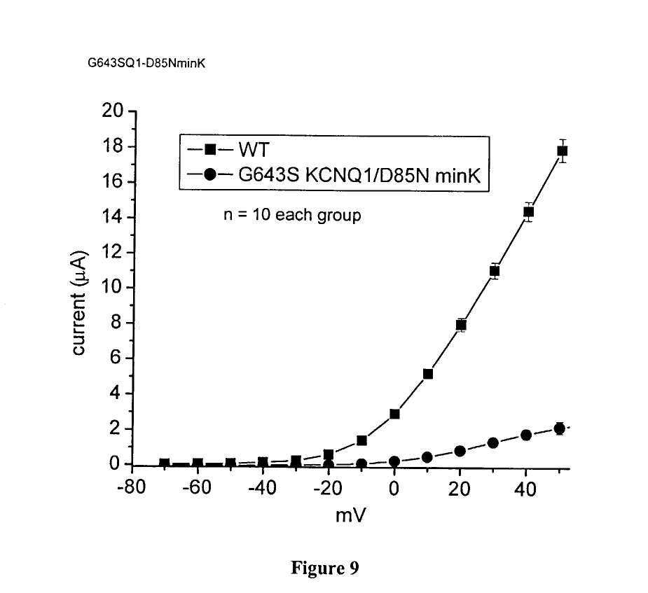

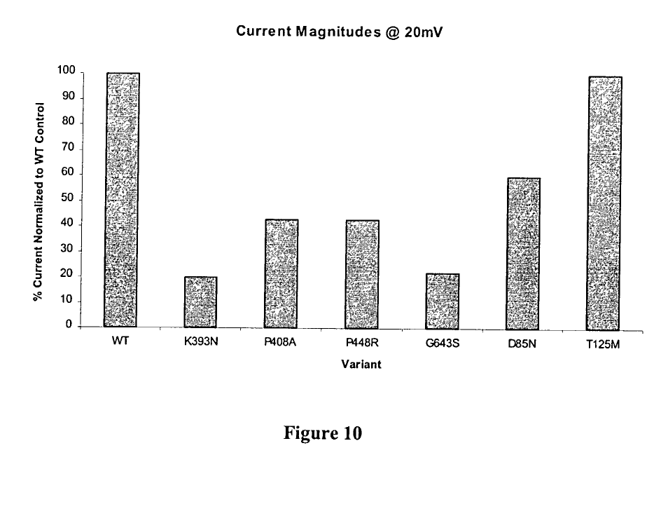

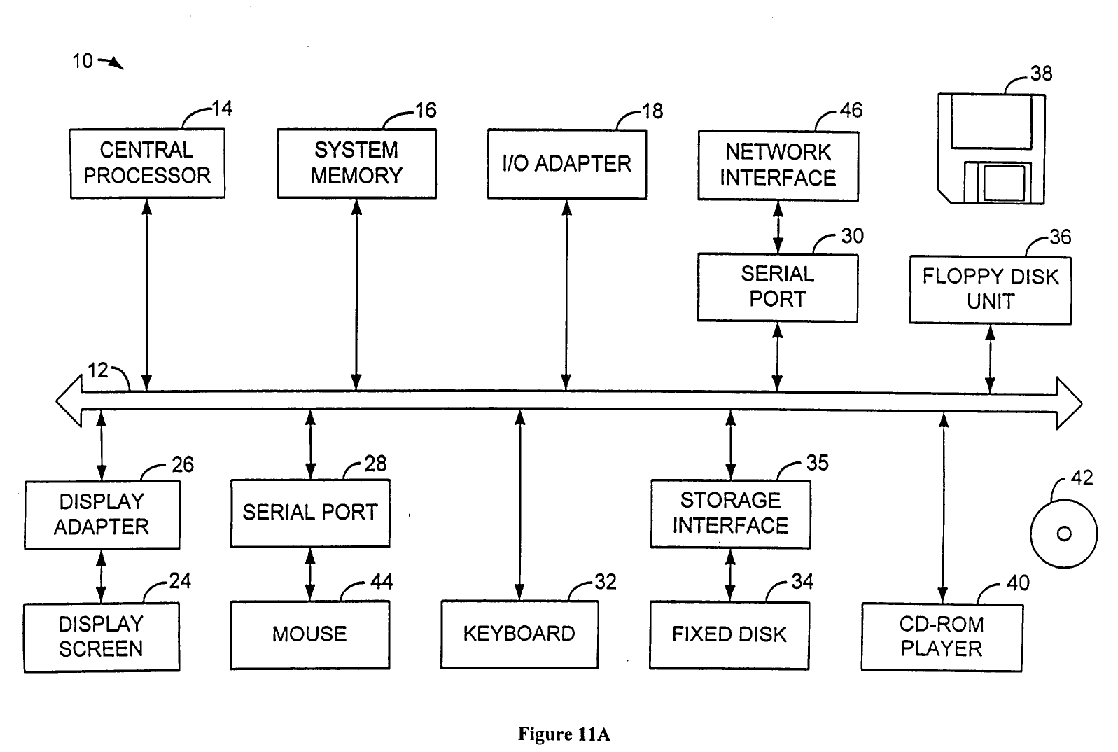

This example demonstrates the current-voltage relationships established for the transfected oocytes. Current-voltage (I-V) relationships were determined and are shown in (FIGS. 2, 3, 6 and 9). This is a measure of current amplitude as a function of test voltage. Currents were measured using 5 sec pulses to potentials ranging from −70 to +50 mV for wildtype channels and −70 to +80 mV for variant channels (to account for shift in gating explained below). K393N KCNQ1 has its own set of controls because this variant was evaluated in a second batch of oocytes. KCNQ1 mutations cause a rightward shift in the I-V relations. Current magnitudes were reduced by more than 50% at any given potential (FIG. 10). The D85N KCNE1 mutation caused a similar, but less dramatic shift in the voltage dependence of current activation, and the T125M KCNE1 variant had no affect (FIG. 5). A G643S KCNQ1/D85 N KCNE1 double mutant reduced current to almost zero indicating that the two mutations act synergistically rather than additively (FIG. 9). This result indicates that patients carrying both mutations are at much greater risk of ion channel disease than patients carrying either of the mutations alone.

### Example 2

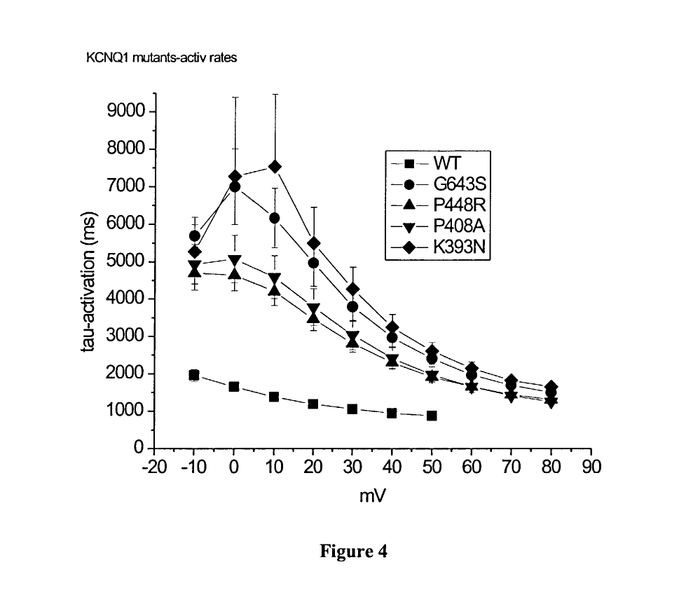

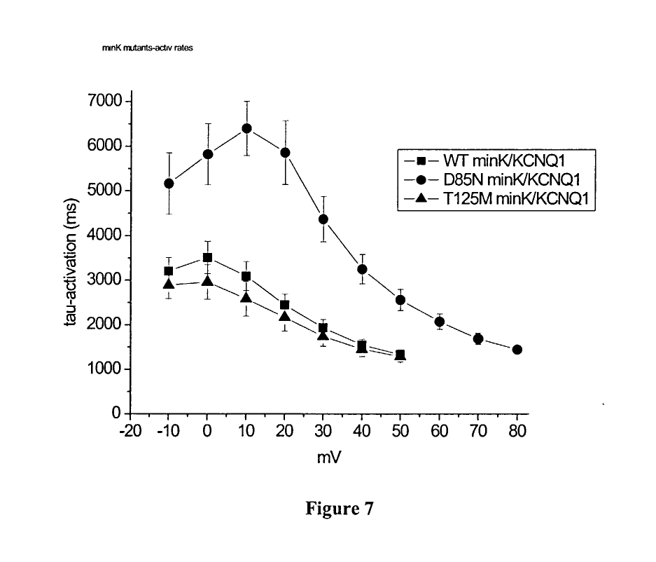

This example demonstrates the activation rate relationships established for the transfected oocytes. Activation rates (FIGS. 4 and 7) were determined by plotting time constants (tau) for activation vs test potential. Time constants describing ion channel kinetic properties are calculated by fitting curves generated from an IV determination. Hille, Ionic Channels in Excitable Membranes. 2nd ed. Sinauer Associates, Sunderland, Mass., p. 607 (1992). This is a measure of how fast channels open from the closed state. A slower time constant would decrease repolarizing current during an action potential. All mutants except T125M KCNE1 slowed the rate of activation.

### Example 3

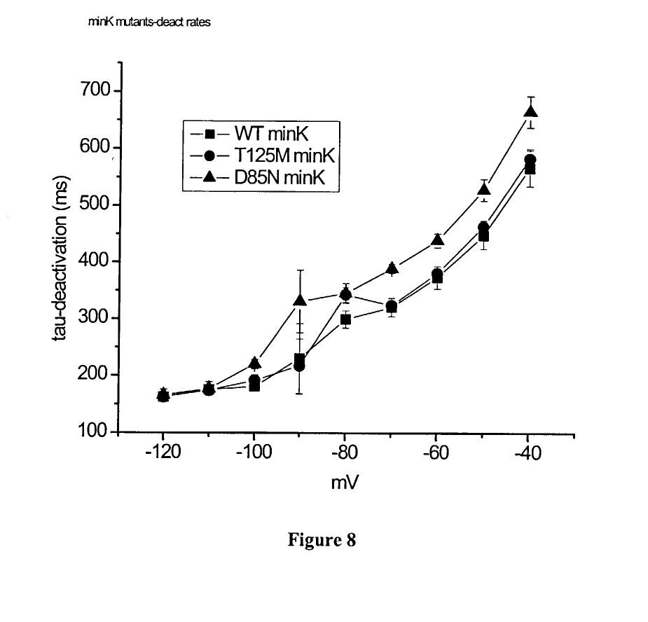

This example demonstrates the deactivation rate relationships established for the transfected oocytes. Deactivation rates (FIGS. 5 and 8) were determined by plotting time constants (tau) for deactivation vs test potential. This is a measure of how fast channels close from an open state. A faster time constant would cause a decrease in current. Deactivation was unchanged for all mutants tested except D85N in which deactivation was reduced. Therefore reduced current through the potassium ion channel due to the mutations in KCNQ1 and KCNE1 is due to slower activation during an action potential.

The results obtained by the methods described in all three examples demonstrate that the variant forms of KCNQ1 and KCNE1 of the present invention have functional effects in reducing net outward repolarizing currents in the potassium channel encoded by these genes. Therefore, the variant forms are correlated with the presence of ion channel disease or susceptibility thereto.

It is to be noted that the term “a” or “an” entity refers to one or more of that entity, including mixtures of the entities of two or more of the entities. As such, the terms “a” (or “an”), “one or more” and “at least one” are used interchangeably herein. It is also to be noted that the terms “comprising,” “including,” and “having” have been used interchangeably.

While various embodiments of the present invention have been described in detail, it is apparent that modifications and adaptations of those embodiments will occur to those skilled in the art. However, it is to be expressly understood that such modifications and adaptations are within the spirit and scope of the present invention, as set forth in the following claims.

[image]

[image]

What is claimed is:

1. A method of determining an ion channel disease genotype of an individual, comprising analyzing a nucleic acid sample from the individual for the presence of at least one mutation indicative of decreased ion channel conductivity that results in an amino acid change selected from the group consisting of:
a. a lysine residue to an asparagine residue at amino acid position 393 of the KCNQ1 protein,
b. a proline residue to an alanine residue at amino acid position 408 of the KCNQ1 protein,
c. a proline residue to an arginine residue at amino acid position 448 of the KCNQ1 protein, and
d. a glutamic acid residue to a serine residue at amino acid position 643 of the KCNQ1 protein.

2. The method of claim 1, wherein the at least one mutation indicative of decreased ion channel conductivity is selected from the group consisting of:
a. the substitution of a thiamine for a guanine at position 1179 of the KCNQ1 coding sequence,
b. the substitution of a guanine for a cytosine at position 1222 of the KCNQ1 coding sequence,
c. the substitution of a guanine for a cytosine at position 1343 of the KCNQ1 coding sequence, and
d. the substitution of an adenine for a guanine at position 1927 of the KCNQ1 coding sequence.

3. The method of claim 1, comprising the further step of:
analyzing the nucleic acid sample for the presence of a mutation that results in an amino acid change from an aspartic acid residue to an asparagine residue at amino acid position 85 of the KCNE1 protein.

4. The method of claim 3, wherein the mutation encoding an asparagine residue at amino acid position 85 of the KCNE1 protein is the substitution of an adenine for a guanine at position 671 of the KCNE1 coding sequence.

5. The method of claim 1, wherein the step of analyzing is selected from the group consisting of differential primer extension, allele-specific probe hybridization, allele-specific amplification, direct sequencing, denaturing gradient gel electrophoresis, and, single strand conformational polymorphism analysis.

6. The method of claim 1, wherein the individual has or is suspected of having an ion channel disease selected from the group consisting of long QT syndrome, cardiac arrhythmias, epilepsy, hearing loss, SIDS, SUDEP, SUDS post-myocardial infarction complications, and acquired sudden death syndrome.

7. The method of claim 1, wherein the individual is a lineal descendant of an individual who has or is suspected of having an ion channel disease selected from the group consisting of long QT syndrome, cardiac arrhythmias, epilepsy, hearing loss, SIDS, SUDEP, SUDS post-myocardial infarction complications, and acquired sudden death syndrome.

8. The method of claim 3, wherein the analyzing step comprises subjecting a nucleic acid sample from the individual to amplification conditions in the presence of a pair of primers, wherein one of the primers comprises at least twelve nucleotides and has a sequence comprising a sequence selected from the group consisting of:
a. the sequence immediately adjacent to position 1179 of SEQ ID NO: 2 and including either a thiamine or a guanine at position 1179 of SEQ ID NO: 2 as the terminal 3′ base of the primer;
b. the sequence immediately adjacent to position 1179 of the complement of SEQ ID NO: 2 and including either an adenine or a cytosine at position 1179 of the complement of SEQ ID NO: 2 as the terminal 3′ base of the primer;
c. the sequence immediately adjacent to position 1222 of SEQ ID NO: 2 and including either a guanine or a cytosine at position 1222 of SEQ ID NO: 2 as the terminal 3′ base of the primer;
d. the sequence immediately adjacent to position 1222 of the complement of SEQ ID NO: 2 and including either a cytosine or a guanine at position 1222 of the complement of SEQ ID NO: 2 as the terminal 3′ base of the primer;
e. the sequence immediately adjacent to position 1343 of SEQ ID NO: 2 and including either a guanine or a cytosine at position 1343 of SEQ ID NO: 2 as the terminal 3′ base of the primer;
f. the sequence immediately adjacent to position 1343 of the complement of SEQ ID NO: 2 and including either a cytosine or a guanine at position 1343 of the complement of SEQ ID NO: 2 as the terminal 3′ base of the primer;
g. the sequence immediately adjacent to position 1927 of SEQ ID NO: 2 and including either an adenine or a guanine at position 1927 of SEQ ID NO: 2 as the terminal 3′ base of the primer;
h. the sequence immediately adjacent to position 1927 of the complement of SEQ ID NO: 2 and including either a thiamine or a cytosine at position 1927 of the complement of SEQ ID NO: 2 as the terminal 3′ base of the primer;
i. the sequence immediately adjacent to position 671 of SEQ ID NO: 5 and including either an adenine or a guanine at position 671 of SEQ ID NO: 5 as the terminal 3′ base of the primer; and
j. the sequence immediately adjacent to position 671 of the complement of SEQ ID NO: 5 and including either a thiamine or a cytosine at position 671 of the complement of SEQ ID NO: 5 as the terminal 3′ base of the primer.

9. An isolated KCNQ1 nucleic acid molecule comprising a nucleic acid sequence selected from the group consisting of: (a) SEQ ID NO: 6, (b) SEQ ID NO: 7, (c) SEQ ID NO: 8, (d) SEQ ID NO: 9 and (e) a nucleic acid sequence that is fully complementary to a nucleic acid sequence of (a)-(d); wherein said isolated nucleic acid molecule is less than about 5 kilobases in length.

10. The isolated KCNQ1 nucleic acid molecule of claim 9, wherein said nucleic acid molecule is less than about 70 nucleotides in length.

11. The isolated nucleic acid molecule of claim 10, wherein said isolated nucleic acid molecule is a probe of 100 or fewer nucleotides.

12. The isolated nucleic acid molecule of claim 10, wherein said probe comprises 50 or fewer nucleotides.

13. The isolated nucleic acid molecule of claim 10, wherein said probe is conjugated to a detectable marker.

14. An array of oligonucleotides comprising at least one probe of claim 10.

15. An isolated nucleic acid molecule comprising at least one base variation from that of an ion channel associated gene sequence comprising a polymorphic nucleotide identified in Table 4 and at least 20 other bases of the ion channel associated gene identified in Table 4 with said polymorphic nucleotide; wherein said isolated nucleic acid molecule is less than about 5 kilobases in length.

16. An isolated nucleic acid molecule comprising at least one base variation from that of an ion channel associated gene sequence comprising a polymorphic nucleotide identified in Table 5 and at least 20 other bases of the ion channel associated gene identified in Table 5 with said polymorphic nucleotide; wherein said isolated nucleic acid molecule is less than about 5 kilobases in length.
